(19) United States
(12) Patent Application Publication
(10) Pub. No.: US 2023/0346705 A1
(43) Pub. Date: Nov. 2, 2023

(54) NANOPARTICLES FOR TREATING OR PREVENTING A CARDIOMYOPATHY AND ANTHRACYCLINE-CYTOTOXICITY, AND THEIR ADMINISTRATION AS AN AEROSOL

(71) Applicants: INSTITUT NATIONAL DE LA SANTE ET DE LA RECHERCHE MEDICALE (INSERM), Paris (FR); CENTRE NATIONAL DE LA RECHERCHE SCIENTIFIQUE (CNRS), Paris (FR); UNIVERSITE PAUL SABATIER TOULOUSE III, Toulouse Cedex 9 (FR); CHROMALYS, Toulouse (FR)

(72) Inventors: Jeanne MIALET-PEREZ, Toulouse Cedex 4 (FR); Séverine LECHEVALLIER, Verniolle (FR); Yohan SANTIN, Toulouse Cedex 4 (FR); Marc Raoul Joseph VERELST, Toulouse (FR)

(21) Appl. No.: 18/044,407

(22) PCT Filed: Sep. 7, 2021
(86) PCT No.: PCT/EP2021/074624

§ 371 Date: Mar. 8, 2023

(30) Foreign Application Priority Data
Sep. 10, 2020 (EP) 20306004.1
May 26, 2021 (EP) 21305682.3

(51) Int. Cl. A61K 9/16; A61K 9/00; A61K 31/704; A61P 9/04

(52) U.S. Cl. CPC A61K 9/1647; A61K 9/0078; A61K 31/704; A61P 9/04; A61K 9/1611

(57) ABSTRACT

Anthracyclines such as doxorubicin are chemotherapeutic molecules are also widely incorporated in many chemotherapy protocols. However, their clinical use is still limited by time- and dose-dependent cardiotoxicity. Herein the inventors have determined the therapeutic potential of acidic nanoparticles (NPs) in doxo-treated cardiac cells. In particular, they have identified a set of grafted nanoparticles as non-toxic and which rapidly internalize into lysosomes in cardiac cells. Such NPs improve lysosomal acidification and autophagic flux blockade caused by bafilomycin A1, chloroquine and doxorubicin, resulting in reduced oxidative stress, preserved mitochondrial integrity and improved cell survival. Thus, the invention relates to a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle; for use in a method for treating or preventing a cardiomyopathy or anthracycline cytotoxicity.

### FIELD OF THE DISCLOSURE

The invention relates to the field of nanoparticles; in particular for use as a medicament, and more particularly for treating or preventing a cardiomyopathy and anthracycline-cytotoxicity.

### BACKGROUND OF THE DISCLOSURE

Autophagy is a subcellular process for lysosome-mediated turnover of damaged mitochondria or proteins, acting as a major cytoprotective mechanism in stress conditions. Disturbance of the autophagy pathway underlines the development of various pathologies in the field of cancer, neurodegeneration, skeletal myopathies and cardiac diseases so its modulation has broad therapeutic interest. Autophagy proceeds through sequential steps, consisting in the formation and maturation of autophagosomes and then fusion with lysosomes to degrade unwanted cytoplasmic constituents. Although the importance of lysosomes has long been undervalued, it is now clear that the efficiency of autophagy strongly relies on the capacity of these organelles to degrade the delivered cargo (Dikic and Elazar; 2018; “Mechanism and medical implications of mammalian autophagy”; Nat. Rev. Mol. Cell. Bio.; 19(6):349-364).

In the heart, lysosomes are now recognized as major players of the regulation of autophagy in baseline conditions and during adaptation to stress (Wang and Robbins; 2014; “Proteasomal and lysosomal protein degradation and heart disease”; J. Mol. Cell. Cardiol.; 71:16-24). Lysosomal defects have also been recently described in oxidative stress-related cardiac damage due to the overexpression of monoamine oxidase-A in the heart (Santin et al.; 2016; “Oxidative stress by Monoamine Oxidase-A Impairs Transcription Factor EB Activation and Autophagosome Clearance, Leading to Cardiomyocyte Necrosis and Heart Failure; Antioxid Redox Signal; 25(1):10-27).

Lysosomal alteration seems to be a common factor of many drug-induced cardiomyopathy, which remains an important cause of heart disease (Feenstra et al. 1999). More than 70 cases of cardiotoxicity have been reported in which the anti-malarial molecule chloroquine has primarily been implicated.

Anthracyclines such as doxorubicin are chemotherapeutic molecules widely incorporated in many chemotherapy protocols. However, their clinical use is still limited by time- and dose-dependent cardiotoxicity. In particular, doxorubicin (doxo) is an anthracycline used as an effective cancer chemotherapeutic, which is also involved in a frequent cardiac disease such as doxorubicin-induced cardiomyopathy (Bartlett et al.; 2017 “Doxorubicin impairs cardiomyocyte viability by suppressing transcription factor EB expression and disrupting autophagy”; The Biochemical journal; 473(21):3769-3789).

In addition to its role on DNA damage and mitochondrial dysfunction, doxorubicin also blocks autophagic flux in cardiomyocytes by impairing lysosomal acidification. At present, strategies designed to reduce anthracycline toxicity, or even cardiomyopathies in general, are still lacking.

Nevertheless, it is still unknown whether anthracyclin-induced (i.e. doxorubicin-induced) mitochondrial damage and cell dysfunction could be attenuated by ameliorating lysosomal function in cardiac cells.

In this context, an original approach relies on the use of nanoparticles prepared from biodegradable and biocompatible polymers, which have the advantage to be delivered to lysosomes through endocytic pathways. More specifically, polymeric poly(lactic-co-glycolic acid) nanoparticles have demonstrated some ability to acidify lysosomes of neurons and retinal epithelial cells (Bourdenx et al.; 2016; “Nanoparticles restore lysosomal acidification defects: Implications for Parkinson and other lysosomal-related diseases; Autophagy; 12(3):472-483).

At present, despite remarkable advances in the use of nanoparticles in the cancer field, very few preclinical tests have been reported for nanomedicine applied to the treatment of cardiovascular diseases. Indeed, limited cardiac specificity of systemically-administered drugs severely hampers therapeutic efficacy and increases adverse side effects.

Hence, the prospective benefits of nanoparticles in cardiac cells with lysosomal alteration have never been assessed, together with their role on autophagic flux and cell function in a pathological context such as doxorubicin toxicity.

Hence, there is a strong need to identify and validate new modulators of autophagy able to restore lysosomal function.

In particular, there remains a need for active agents for treating or preventing anthracycline-toxicity, or an associated condition; in particular doxorubicin-toxicity.

There also remains a need for active agents for treating or preventing a cardiomyopathy. In particular, there remains a need for treating or preventing a drug-induced cardiomyopathy such as anthracycline-induced cardiomyopathies.

The invention has for purpose to meet the above-mentioned needs.

### SUMMARY

According to a first main embodiment, the invention relates to a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle; for use in a method for treating or preventing a cardiomyopathy. In particular, the cardiomyopathy can be a drug-induced cardiomyopathy, such as an anthracycline-induced cardiomyopathy.

According to a second main embodiment, the invention relates to a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle; for use in a method for treating or preventing anthracycline-toxicity.

According to a third main embodiment, the invention relates to a pharmaceutical composition comprising a biocompatible and biodegradable nanoparticle, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle; for use in a method for treating or preventing a cardiomyopathy or an anthracycline-toxicity.

According to a fourth main embodiment, the invention relates to a kit comprising:
a first part comprising a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle; and
a second part comprising at least one anthracycline.

According to a fifth main embodiment, the invention relates to a pharmaceutical aerosol composition comprising a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle.

According to a sixth main embodiment, the invention relates to a pharmaceutical aerosol composition as defined above; for use as a medicament, and in particular for use in a method for treating or preventing a cardiomyopathy and/or an anthracycline-toxicity.

According to a seventh main embodiment, the invention relates to an aerosol generating device comprising: a pharmaceutical composition comprising biocompatible and biodegradable nanoparticles having a diameter of 100 nm or less, wherein the nanoparticles are selected from: poly(lactic-co-glycolic acid) (PLGA) nanoparticles, poly(lactic acid) (PLA) nanoparticles, poly(glutamic acid) (PGA) nanoparticles, polycaprolactone (PCL) nanoparticles, and/or polyester nanoparticles.

### DESCRIPTION OF THE FIGURES

FIG. 1. Characterization of the nanoparticles with and without PLGA. (A) Size distribution histogram of NPs-PLGA. (B) Infrared spectra of nanoparticles (NPs) and nanoparticles grafted with PLGA (NPs-PLGA), respectively as top and bottom curves. The x-axis represents the wavenumber (cm⁻¹) and the y-axis represents transmittance expressed in arbitrary units.

FIG. 2. Cytotoxicity, cellular uptake and lysosomal targeting of NPs-PLGA (A) LDH release and (B) Caspase 3 activity in H9C2 incubated with increasing times and doses of NPs-PLGA (n=4). Each set of data is collected, from left to right, after 1 h, 8 h, 24 h and 48 h at increasing concentrations of NPs-PLGA, with from left to right: 0, 5, 10, 25, 50, 75, 100, 150, and 200 μg/mL for (A) and of 0, 5, 10 and 25 for (B). LDH release and caspase 3 activity are expressed in the y-axis as Fi/CT. (C) Intracellular uptake rate of 25 μg/mL of NPs-PLGA in H9C2 (n=3 & *** means p<0.001 vs control). In the x-axis, time points consist of 0, 5 minutes, 15 minutes, 30 minutes, 1 h, 4 h, 8 h, and 24 h. The percentage of FITC positive cells is indicated in the y-axis.

FIG. 3. NPs-PLGA alleviate Bafilomycin A1-induced lysosomal dysfunction in H9C2. (A-C) H9C2 were pre-incubated with 25 μg/mL of NPs or NPs-PLGA and treated with Bafilomycin A1 (100 nM). (A) Timeline of experimental protocol. (B) Fluorometer measurements of Acridine Orange red staining (n=6). (C) Fluorometer measurements of Lysosensor yellow/blue staining (n=7). Excitation was measured at 360 nm and the ratio of emission 440/540 nm was calculated. Datas are expressed as means+/−sem (*p<0.05, ***p<0.001 vs CTL; #p<0.05, ##p<0.01 vs Baf A1).

FIG. 4. NPs-PLGA mitigate Bafilomycin A1-induced autophagic flux blockade in H9C2. (A) Quantification of LC3 (bottom panel) and p62 (upper panel) ratios to GADPH (n=6). Ratios expressed as FI/CT in the y-axis. (B) Quantification of yellow puncta (autophagosomes or AP—left column for each set of conditions) and red puncta (autolysosomes or AL—right column for each set of conditions) is displayed on histogram (n=6). Data are expressed as means+/−sem (***p<0.001 vs. CTL; #p<0.05 vs Baf A1).

FIG. 5. NPs-PLGA prevent doxorubicin-induced lysosomal and autophagy alteration in H9C2. (A-D) H9C2 were pre-incubated with 25 μg/mL of NPs or NPs-PLGA and treated with doxorubucin (1 μM). (A) Timeline experimental protocol. (B) Fluorometer measurements of Lysosensor yellow/blue staining (n=6). Excitation was measured at 360 nm and the ratio of emission 440/540 nm was calculated. The corresponding lyososomal pH is set in the y-axis, from 5.0 to 6.0. (C) Fluorometer measurements of Cathepsin D activity fluorescence (n=6), expressed as RFU 328/460 nm ratio in the y-axis. (D) Quantification of yellow puncta (autophagosomes or AP—left column for each set of conditions) and red puncta (autolysosomes or AL—left column for each set of conditions) is displayed on histogram (n=6), as a percentage of autophagic vesicles. Datas are expressed as means+/−sem (*p<0.05, **p<0.01, ***p<0.001 vs. CTL; #p<0.05, ##p<0.01 vs. Doxo).

FIG. 6. NPs-PLGA lessen doxorubucin-induced cell dysfunction and death in H9C2. (A-D) H9C2 were pre-incubated with 25 μg/mL of NPs or NPs-PLGA and treated with Doxorubicin (1 μM). (A) ROS levels assessed by fluorometer measurements of DCFDA fluorescence (n=4) as expressed in FI/CT, in the y-axis. (B) Mitochondrial depolarization assessed by fluorometer measurements of JC-1 red aggregates/green monomers ratio (n=6), as expressed in FI/CT in the y-axis. (C-D) Quantifications of p-p53 (C) and cleaved caspase 3 ratios (D) to GAPDH (n=6), as expressed in FI/CT. Datas are expressed as means±sem (*p<0.05, **p<0.01, ***p<0.001 vs CTL; #p<0.05, ##p<0.01 vs Doxo).

FIG. 7. NPs-PLGA prevent Doxorubicin-induced lysosomal, autophagy and cell dysfunction in Neonatal Rat Ventricular Myocytes (NRVMs) (A-D) NRVMs were pre-incubated with 200 μg/mL of NPs or NPs-PLGA and treated with Doxorubicin (1 μM). (A) Fluorometer measurements of Lysosensor yellow/blue staining (n=6). Excitation was measured at 360 nm and the ratio of emission 440/540 nm was calculated. (B) Quantification of yellow puncta (autophagosomes) and red puncta (autolysosomes) for each condition is displayed on histogram (n=6). (C) ROS levels assessed by fluorometer measurements of DCFDA fluorescence (n=6). (D) Caspase 3 activity in NRVMs (n=4). Datas are expressed as means±sem (*p<0.05, **p<0.01, ***p<0.001 vs CTL; #p<0.05, ##p<0.01 vs Doxo).

FIG. 8. NPs-PLGA reacidify dysfunctional lysosomes and restore autophagic flux in Doxorubicin-treated Neonatal Rat Ventricular Myocytes (NRVMs) (A-E) NRVMs were pre-treated with Doxorubicin (250 nM) and incubated with 200 μg/mL of NPs or NPs-PLGA. (A) Timeline of experimental protocol and fluorometer measurements of Acridine Orange red staining (n=4). (B) Fluorometer measurements of Acridine Orange red staining (n=6) (C) Fluorometer measurements of Lysosensor yellow/blue staining (n=8). Excitation was measured at 360 nm and the ratio of emission 440/540 nm was calculated. (D) Quantification of yellow puncta (autophagosomes) and red puncta (autolysosomes) for each condition is displayed on histogram (n=7). (E) Fluorometer measurements of Cathepsin D activity fluorescence (n=5). (F) Mitochondrial ROS levels assessed by fluorometer measurements of mitoSOX fluorescence (n=4). Datas are expressed as means±sem (**p<0.01, ***p<0.001 vs CTL; #p<0.05, ##p<0.01 vs Doxo).

FIG. 9. Effect of MPs-PLGA vs. NPs-PLGA. (A) LDH release in neonatal rat ventricular myocytes (NRVMs) incubated with NPs-PLGA or MPs-PLGA for 24 h (n=4) (B) Intracellular uptake rate of FITC-MPs-PLGA in NRVMs (n=2) (C) Acridine Orange red fluorescence reflecting lysosomal acidification in NRVMs treated with 250 nM of doxorubicin for 24 h and then incubated with NPs-PLGA or MPs-PLGA (n=4). Datas are expressed as means±sem (**p<0.01, ***p<0.001 vs CTL; ##p<0.01 vs Doxo).

FIG. 10. NPs-PLGA alleviate Chloroquin-induced autophagic flux blockade in H9C2. (A-CB) H9C2 were pre-incubated with 25 μg/mL of NPs or NPs-PLGA and treated with Chloroquin for 4 h. (A) Quantifications of LC3 (lower panel) and p62 (upper panel) ratios to GAPDH (n=5). (B) Quantification of yellow puncta (autophagosomes) and red puncta (autolysosomes) for each condition is displayed on histogram (n=5). Datas are expressed as means±sem (*p<0.05, **p<0.01, ***p<0.001 vs CTL; #p<0.05 vs CQ).

FIG. 11. NPs administration via intratracheal nebulization and its effect on autophagic markers. (A) Schematic representation of NPs administration via intratracheal nebulization. After reaching the lungs from the trachea, NPs cross the pulmonary vein and accumulate within the heart. (B) Quantifications of autophagic markers LC3-II (left) and p62 (right) in cardiac homogenates of mice treated or not with Doxorubicin (5 mg/kg i.v.) and NPs-PLGA (0.5 or 1 mg/kg intratracheal), relatively to the expression of a GAPDH control signal in an immunoblot. From left to right, for each x-axis, the following experiments are provided: CT (control), Dox (Doxorubicin), NPs0.5 and NPs1.

FIG. 12. NPs administration via intratrachel nebulization prevents doxorubin-induced cardiac remodeling and impairment of cardiac function in vivo. A Experimental protocol of doxorubicin treatment (4 once-per-week injections of 5 mg/kg) and nanoparticles intra-tracheal nebulization (1 mg/kg right after doxorubicin injection) followed by assessment of cardiac remodeling and function in mice. B Mouse weight one week after the last administration of doxorubicin and nanoparticles highlighting 1/nanoparticles safety on mouse weight in control mice (without doxorubicin) and 2/nanoparticles beneficial effect on the limitation of weight loss in mice treated with doxorubicin. C Heart weight to tibia length ratio after mouse sacrifice showing 1/nanoparticles safety on heart weight in control mice (without doxorubicin) and 2/nanoparticles beneficial effect on the limitation of cardiac atrophy) in mice treated with doxorubicin. D Echocardiographic measurement of cardiac contractility (ejection fraction) one week after the last administration of doxorubicin and nanoparticles highlighting 1/nanoparticles safety on cardiac function in control mice (without doxorubicin) and 2/nanoparticles beneficial effect on the limitation of cardiac contractile dysfunction in mice treated with doxorubicin.

### DETAILED DESCRIPTION OF THE DISCLOSURE

The disclosure relates to a biocompatible and biodegradable nanoparticle having a diameter of 100 nm or less; wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle.

In particular, this therapeutic potential was demonstrated with acidic nanoparticles (NPs) grafted with poly(lactic-co-glycolic acid) (NPs-PLGA) in doxo-treated cardiac cells.

Those PLGA-grafted nanoparticles (NPs-PLGA) were shown to be non-toxic, and to rapidly internalize into lysosomes in cardiac cells. NPs-PLGA improve lysosomal acidification and autophagic flux blockade caused by bafilomycin A1, chloroquine and doxorubicin, resulting in reduced oxidative stress, preserved mitochondrial integrity and improved cell survival.

To our knowledge, those data show for the first time that application of such acidic NPs on cardiac cells improves lysosomal acidity and restores autophagic flux in lysosomal-related cardiomyocyte impairment.

This beneficial effect on lysosomal pH is also observed in a situation of oxidative stress due to the overactivation of the mitochondrial enzyme MAO-A, which has been described to impair the lysosome function and autophagic flux.

Interestingly, our results reveal NPs-PLGA ability to reacidify defective lysosomes and restore lysosomal function when applied after anthracyclin treatment (i.e. doxorubicin). These findings are of great importance as they highlight the role of lysosomal recovery in cardiac improvement and they provide therapeutic solutions against doxorubicin-linked dysfunction. We have used a novel formulation of acidic nanoparticles, NPs grafted with PLGA, that ameliorate lysosomal acidity and autophagix flux in the presence of different lysosomal damaging agents. These findings are important as they provide evidence that autophagy can be ameliorated in cardiac cells, even when it is blocked at a late stage.

Also, the therapeutic potential of such nanoparticles is now confirmed in vivo in a mouse model related to doxorubicin-toxicity, with intratracheal administration and nebulization. In particular, it is shown herein that an aerosol comprising the reported nanoparticles has a pronounced tropism toward the cardiac muscle in vivo, and is thus particularly convenient for clinical use.

In particular, it is shown here that such nanoparticles are efficient in vivo in preventing cardiac toxicity, by preventing cardiac remodeling and impairment of the cardiac function. On the other hand, the administered nanoparticles do not exhibit any particular toxicity on their own.

Overall, the use of acidic polymers such as PLGA grafted onto nanoparticles having a diameter of 100 nm or less in the present study offers several advantages:
(i) it is compatible with aerosol formation and nebulization.
(ii) the size of the nanoparticles is adapted to a rapid and lasting cell incorporation by endocytosis. These data correlate with our results showing that nanoparticles of 200 nm have altered cellular internalization and no functional effect on pH.
(iii) these nanoparticles can be easily labeled with a detectable (i.e. fluorescent) tracer such as fluorescein allowing an easy targeting process study.
(iv) acidic polymers such as PLGA are Food and Drug Administration (FDA)-approved owing to their biodegradability and biocompatibility.
(v) the hydrolysis of acidic polymers such as PLGA slowly releasing acidic functions avoids the dispersion of acids before being incorporated into the targeted cells. In a non-limitative manner, the PLGA polyester can be obtained by means of ring-opening co-polymerization of two different monomers, the cyclic dimers of glycolic acid and lactic acid. This reaction is reversible in aqueous medium by slow hydrolysis which releases the two corresponding organic acids. Thus, acidic polymers and copolymers such as PLGA are likely to be effective and long-lasting against lysosome alkalinisation and dysfunction.

Our results also show that improvement of lysosomal pH correlates with the preservation of mitochondrial function and improvement of cell viability. Without wishing to be bound by theory, the inventors are of the opinion that NPs grafted with acidic polymers, such as NPs-PLGA, have acidic properties, meaning that the PLGA copolymer or polymer which is grafted at their surface is susceptible to provide protons upon hydrolysis in a biological environment, such as the cell. Hence, those nanoparticles improve autophagic flux, which could explain the improvement in mitochondrial quality.

Together, our results show that those acidic nanoparticles are trafficked to the lysosomes and can then improve organelle function when used as preventive or curative tools. They represent attractive and original tools which could provide health benefits in different conditions of lysosomal impairment.

Besides, the observed ability of those acidic nanoparticles to reverse the toxicity of particular compounds such as anthracycline, now provides an incentive to administer them in combination with anthracyclin (i.e. simultaneously or in a sequential manner) in order to limit their detrimental effects; in particular in relation with cardiomyocytes, and more generally cardiac physiology.

Hence, the inventors also provide a therapeutic method comprising a step of administering to a patient in need thereof an aerosol comprising a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle.

Also, the inventors provide a method for reversing the toxicity of compounds, in particular anthracycline-cytotoxicity, and for treating or preventing a cardiomyopathy, comprising a step of administering a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle. Such nanoparticles are preferably administered alone, but may also be co-administered with a cardiomyopathy-inducing drug and/or an anthracycline. For instance, the nanoparticles and the cardiomyopathy-inducing drug or anthracycline can be administered sequentially, or concomitantly (i.e. at the same time, in combination through the same administration route or through two distinct administration routes).

According to a first main embodiment, the invention relates to a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle; for use in a method for treating or preventing a cardiomyopathy. In particular, the cardiomyopathy can be a drug-induced cardiomyopathy, such as an anthracycline-induced cardiomyopathy.

In particular, the cardiomyopathy is a drug-induced cardiomyopathy induced by an anthracycline selected from the list consisting of: daunorubicin, doxorubicin, epirubicin, farmorubicin, idarubicin, mitoxantrone, pixantrone; and their pharmaceutically acceptable salts. More particularly, the cardiomyopathy is a drug-induced cardiomyopathy induced by doxorubicin, or a pharmaceutically acceptable salt thereof.

Alternatively, the cardiomyopathy is a drug-induced cardiomyopathy induced by an amino-4-quinoleine or a derivative thereof; in particular induced by chloroquine, hydroxychloroquine, or a pharmaceutically acceptable salt thereof.

According to a second main embodiment, the invention relates to a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle; for use in a method for treating or preventing anthracycline-toxicity.

According to a third main embodiment, the invention relates to a pharmaceutical composition comprising a biocompatible and biodegradable nanoparticle as defined above, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle; for use in a method for treating or preventing a cardiomyopathy or an anthracycline-toxicity.

According to a fourth main embodiment, the invention relates to a kit comprising:
a first part comprising a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle; and
a second part comprising at least one anthracycline.

According to a fifth main embodiment, the invention relates to a pharmaceutical aerosol composition comprising a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle.

According to a sixth main embodiment, the invention relates to a pharmaceutical aerosol composition as defined above; for use as a medicament, and in particular for use in a method for treating or preventing a cardiomyopathy and/or an anthracycline-toxicity.

According to a seventh main embodiment, the invention relates to an aerosol generating device comprising: a pharmaceutical composition comprising biocompatible and biodegradable nanoparticles having a diameter of 100 nm or less, wherein the nanoparticles are selected from: poly(lactic-co-glycolic acid) (PLGA) nanoparticles, poly(lactic acid) (PLA) nanoparticles, poly(glutamic acid) (PGA) nanoparticles, polycaprolactone (PCL) nanoparticles, and/or polyester nanoparticles.

### Definitions

As used herein, “treating” means any manner in which one or more of the symptoms of a disease or disorder are ameliorated or otherwise beneficially altered. As used herein, amelioration of the symptoms of a particular disorder refers to any lessening of the symptoms, whether permanent or temporary, lasting or transient, that can be attributed to or associated with treatment by the compositions and methods of the present invention. Accordingly, the expression “treating” may include “reversing partially or totally the effect” of a given condition, or even “curing” when permanent reversal is considered.

As used herein, “preventing” encompasses “reducing the likelihood of occurrence” and “reducing the likelihood of re-occurrence” and “delaying the likelihood of occurrence or re-occurrence”.

The terms “effective amount” and “effective to treat,” as used herein, refer to an amount or a concentration of one or more of the compositions described herein utilized for a period of time (including acute or chronic administration and periodic or continuous administration) that is effective within the context of its administration for causing an intended effect or physiological outcome.

As used herein, the term “subject” or “patient” may encompass an animal, human or non-human, rodent or non-rodent. Veterinary and non-veterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs.

As used herein, the singular form “a”, “an” and “the” include plural references unless the context clearly dictates otherwise. For example, the term “a pharmaceutically acceptable carrier” encompasses a plurality of pharmaceutically acceptable carriers, including mixtures thereof.

As used herein, «a plurality of» may thus include «two» or «two or more».

As used herein, «comprising» may include «consisting of».

As used herein, the term “biocompatible” is meant to refer to compounds (e.g. nanoparticles) which do not cause a significant adverse reaction in a living animal when used in pharmaceutically relevant amounts.

As used herein, a “pharmaceutically acceptable carrier” is intended to include any and all carrier (such as any solvents, dispersion media, coatings, antibacterial and antifungal agents, isotonic and absorption delaying agents, and the like) which is compatible with pharmaceutical administration. The use of such media and agents for pharmaceutically active substances are known. Except insofar as any conventional media or agent is incompatible with the active compound, such media can be used in the compositions of the invention.

As used herein, the term “nanoparticles” is meant to refer to particles having an average size (such as a diameter, for spherical or nearly spherical nanoparticles) of 100 nanometres (nm) in size or less. The “diameter” is typically defined herein as the “Feret diameter” or as the “hydrodynamic diameter”. The hydrodynamic diameter/size is generally measured by dynamic laser light scattering (DLS), in demineralized water, at a physiological pH, used for biological evaluation as well as in vitro and in vivo experiments.

On the other hand, the Feret diameter corresponds to the distance between the two parallel planes restricting the object (i.e. the nanoparticle) perpendicular to the observed direction. Hence, the Feret diameter is generally measured by transmission electron microscopy in demineralized water, as described in the Material &Methods section. In a general manner, the hydrodynamic diameter is slightly above the Feret diameter.

As a general reference, the average size (or “diameter”) is most preferably determined, as the Feret diameter, by transmission electron microscopy in demineralized water, at a physiological pH.

Even though spherical nanoparticles are particularly considered in the context of the invention, it will be understood herein that the term “nanoparticle” is not meant to refer exclusively to one type of shape. Accordingly, this term may also encompass other shapes, selected from: spherical nanoparticles, rod-shaped nanoparticles, vesicle-shaped nanoparticles, and S-shaped worm-like particles as described in Hinde et al. (“Pair correlation microscopy reveals the role of nanoparticle shape in intracellular transport and site of drug release”; Nature nanotechnology; 2016) as well as other morphologies such as nanoflower, raspberry, and core-shell nanoparticles.

In some embodiments, the nanoparticles have an average size (or “diameter”) of about 1-100 nm, e.g., about 10-70 nm, e.g., about 10-60 nm, or about 20-30 nm. The polymer component in some embodiments can be in the form of a coating, e.g., about 5 to 20 nm thick or more. According to a most preferred embodiment, the nanoparticles have an average size (or «diameter») of less than about 50 nm. Thus, a nanoparticle (or population thereof) having an average size (or «diameter») of less than about 100 nm encompasses nanoparticles (or populations thereof) having an average size (or «diameter») of less than about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, and 3 nm.

As used herein, the term “linked to”, such as in “a ligand linked to the nanoparticles” may refer either to a covalent link or to a non-covalent link. In a non-limitative manner, such non-covalent interactions may occur due to electrostatic interactions, Van der Walls forces, π-effects, and hydrophobic effects. Alternatively, covalent-interactions occur as a consequence of the formation of a covalent bond, such as the coupling of a compound of interest (i.e. a cardiomyopathy-inducing drug and/or an anthracycline), with a functional (reactive) chemical group at the surface of the nanoparticle. As used herein, the terms “functionalized by” and “linked to” and “surface-grafted” can be used interchangeably.

As used herein, a “polyester nanoparticle” refers to a nanoparticle functionalized with polyester polymers. Polyesters are polymers that are characterized by the presence of an ester functional group in their main chain. They are generally obtained through the reaction of a diol with a diacid, for instance through a polycondensation reaction. Polyesters can be characterized based on the composition of their main chain (aliphatic/semi-aromatic/aromatic) and whether they are (homo)polymers or co-polymers. Such polyesters include poly(lactic-co-glycolic acid) (PLGA), poly-lactic acid (PLA), poly-glutamic acid (PGA), polycaprolactone (PCL), polyhydroxyalkanoate (PHA), polyhydroxybutyrate (PHB), polyethylene adipate (PEA), polybutylene succinate (PBS), polybutylene succinate co-adipate (PBSA), polybutylene adipate co-terephtalate (PBAT), poly(3-hydroxybutyrate-co-3-hydroxyvalerate) (PHB V), polyethylene terephthalate (PET), polybutylene terephthalate (PBT), polytrimethylene terephthalate (PTT), polyethylene naphthalate (PEN). Such polyesters also include ethylene vinyl acetate polymer (EVA), poly(L-lactic acid) (PLLA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide).

Also, aliphatic polyesters can be assembled from lactones. Hence, such polyester nanoparticles include, in a non-limitative manner, those obtained from the polymerisation or co-polymerisation of poly (lactic acid), poly (lactide-co-glycolide) and/or poly (ε-caprolactone) (PCL).

As used herein, “PLGA” or “PLG” or “poly(lactic-co-glycolic acid)” refers to a block-copolymer or copolymer that can be prepared at different ratios between its constituent monomers, lactic acid (LA) and glycolic acid (GA). Depending on the ratio of lactide to glycolide used for the polymerization, different forms of PLGA can be obtained. However, it will be readily understood by the skilled in the art that all forms of PLGA are envisioned, unless stated otherwise. For instance, PLGA may refer to a copolymer comprising about 50% of lactic acid and 50% of glycolic acid as a molar ratio of monomers; or alternatively a copolymer comprising about 75% of lactic acid and 25% of glycolic acid as a molar ratio.

As used herein, a “PLGA nanoparticle”, a “(poly-lactic acid) (PLA) nanoparticle”, a “(poly-glutamic acid) (PGA) nanoparticle” are nanoparticles functionalized with, respectively, PLGA, PLA or PGA.

As used herein, a “polycaprolactone nanoparticle” refers to a nanoparticle functionalized with polycaprolactones (PCL).

However, those nanoparticle definition do not mean that the nanoparticle may not also include other polymeric material. Hence, those terms may encompass nanoparticles which are functionalized essentially or exclusively with PLGA/PLA/PGA/polyesters, but also nanoparticles which are functionalized with a mixture of polymeric material, which may or may not belong to the category of PLGA/PLA/PGA/polyesters.

As used herein, an “anthracycline” refers to a family of antibiotics often used in cancer chemotherapy. The anthracycline group of compounds, and its pharmaceutically acceptable salts and derivatives, can be broadly defined as a group having a planar anthraquinone chromophore that can intercalate between adjacent base pairs of DNA. This chromophore is generally linked to a daunosamine sugar moiety. However, the term is not limited to naturally-occurring anthracyclines, such as those isolated from bacterial species, but also encompasses its synthetic and semisynthetic derivatives. In a non-exhaustive manner, compounds belonging to the anthracycline group of compounds can be selected from the list consisting of: daunorubicin, doxorubicin, epirubicin, farmorubicin, idarubicin, mitoxantrone, pixantrone; and their pharmaceutically acceptable salts.

As used herein an “amino-4-quinoleine or derivative thereof” refers to therapeutic class of compounds, which are generally endowed with anti-malarial properties, and which includes chloroquine, hydroxychloroquine, amodiaquine, or a pharmaceutically acceptable salt thereof.

As used herein, the term “cardiomyopathy” refers to any group of diseases that affects the heart muscle, such as acute cardiomyopathy and non-acute cardiomyopathy; which includes in particular hypertrophic cardiomyopathy, dilated cardiomyopathy, restrictive cardiomyopathy, arrhythmogenic right ventricular dysplasia, and takotsubo cardiomyopathy. Cardiomyopathies can be classified as primary cardiomyopathies or secondary cardiomyopathies, based on whether the disease is intrinsic or extrinsic. Hence a cardiomyopathy may also be selected from a group consisting of: hypertrophic cardiomyopathy, Arrhythmogenic right ventricular cardiomyopathy (ARVC), LV non-compaction, Ion Channelopathies, Dilated cardiomyopathy (DCM), Restrictive cardiomyopathy (RCM), Stress cardiomyopathy, Myocarditis, inflammation of and injury to heart tissue due in part to its infiltration by lymphocytes and monocytes, Eosinophilic myocarditis, inflammation of and injury to heart tissue due in part to its infiltration by eosinophils, Ischemic cardiomyopathy, Fabry's disease-related cardiomyopathy, hemochromatosis-related cardiomyopathy, Endomyocardial fibrosis, Hypereosinophilic syndrome-related cardiomyopathy, diabetes mellitus-related cardiomyopathy, hyperthyroidism-related cardiomyopathy, acromegaly-related cardiomyopathy, Noonan syndrome's related cardiomyopathy, muscular dystrophy-related cardiomyopathy, Friedreich's ataxia-related cardiomyopathy, Obesity-related cardiomyopathy.

This term further encompasses drug-induced (e.g. iatrogenic) cardiomyopathies, and in particular those induced by anthracyclines and pharmaceutically acceptable salts thereof, and/or any one drug which is prone to provoke or aggravate or disturb the heart function. Hence the term further encompasses a cardiomyopathy induced by an amino-4-quinoleine or a derivative thereof; in particular induced by chloroquine, hydroxychloroquine or a pharmaceutically acceptable salt thereof.

As used herein, the terms “targeting moiety” and “targeting agent” are used interchangeably and are intended to mean any agent, such as a functional group, that serves to target or direct the nanoparticle to a particular location or association (e.g., a specific binding event). Thus, for example, a targeting moiety may be used to target a molecule to a specific target protein or enzyme, or to a particular cellular location, or to a particular cell type, to selectively enhance accumulation of the nanoparticle. Suitable targeting moieties include, but are not limited to, polypeptides, nucleic acids, carbohydrates, lipids, hormones including proteinaceous and steroid hormones, growth factors, receptor ligands, antigens and antibodies, and the like.

As used herein, the term “aerosol” or “aerosol composition” refers broadly to any suspension-type or solution-type aerosol, which comprises or consists of a suspension of solid or liquid particles (i.e. nanoparticles according to the invention) in the air or another gaz (i.e. a propellant for propellant-based aerosol generation devices). Such aerosols can generally be defined for the present purpose as colloidal systems consisting of—very finely divided liquid droplets dispersed in and surrounded by a gas. The droplets in the aerosols typically have a size less than about 50 microns in diameter.

As used herein, the term “suspension aerosol formulation” refers to a formulation in which the product (i.e. the nanoparticle according to the invention or a composition thereof) is in particulate form and is substantially insoluble in the propellant or mixture of propellant and solvent.

As used herein, the term “solution aerosol” refers to a two phase system consisting of the product (i.e. the nanoparticle according to the invention or a composition thereof) in a propellant, a mixture of propellants, or a mixture of propellant and solvent.

As used herein, the term “propellant” refers to any chemical that is compatible with a pharmaceutical use, and which has a vapor pressure greater than atmospheric pressure at 40° C. (105° F.). In a non-limitative manner, types of propellants commonly used in pharmaceutical aerosols include chlorofluorocarbons (CFC), hydrocarbons, hydrochlorofluorocarbons and hydrofluorocarbons, and compressed gases.

Nanoparticles of the Invention

A nanoparticle of the invention is a biocompatible and biodegradable nanoparticles having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle.

In particular, a nanoparticle of the invention is a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, or a polycaprolactone (PCL) nanoparticle.

More particularly, the nanoparticle of the invention is a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, or a poly(glutamic acid) (PGA) nanoparticle.

Preferably, nanoparticles of the invention are biocompatible and biodegradable poly(lactic-co-glycolic acid) (PLGA) nanoparticles having a diameter of 100 nm or less.

Preferably, a nanoparticle of the invention may even have a diameter of less than 50 nm.

A nanoparticle may be a silica-based nanoparticle (SiNP). When the nanoparticle is a SiNP, it is generally obtained (essentially or exclusively) from amorphous silica instead of crystalline silica; although the later can also be used.

Other embodiments of the nanoparticles (NPs) which are relevant in the context of the invention are described hereafter.

The nanoparticles which are particularly considered, and useful in the methods and compositions described herein, are made of materials that are biocompatible e.g. do not cause a significant adverse reaction in a living animal when used in pharmaceutically relevant amounts; optionally feature functional groups to which a binding moiety can be covalently or non-covalently attached, exhibit low non-specific binding of interactive moieties to the nanoparticle; and are stable in solution, e.g., the nanoparticles do not precipitate.

A number of biocompatible nanoparticles are known in the art, e.g., organic or inorganic nanoparticles. Liposomes, dendrimers, carbon nanomaterials and polymeric micelles are examples of organic nanoparticles.

Examples of biocompatible nanoparticles include those including ZnO, Al₂O₃, HfO₂, Ca₁₀(PO₄)₆, CaF₂, NaYF₄, or a mixture thereof.

Magnetic nanoparticles can also be used, e.g., spherical nanocrystals of about 20 nm with a Fe2+ and/or Fe3+ core surrounded by a polymeric material (i.e. PLGA molecules).

Nanoparticles of the invention are preferably poly(lactic-co-glycolic acid (PLGA) nanoparticles. According to exemplary embodiments, nanoparticles of the invention essentially comprise or even exclusively comprise polyesters, such as PLGA, PLA, PGA and/or PCL; or even exclusively PLGA. However, different forms of copolymer (ex. PLGA) can be obtained by using the varied ratio of lactide to glycolide (lactide:glycolide) during polymerization reaction e.g. PLGA 50:50 (refers to a copolymer which comprises of 50% lactic acid and 50% glycolic acid), PLGA 75:25 (refers to a copolymer which comprises of 75% lactic acid and 25% glycolic acid), PLGA 65:35, PLGA 80:20, PLGA 85:15, and so on. All forms of PLGA are considered as suitable for the invention. In particular, the nanoparticles are functionalized with PLGA 50:50.

Depending upon the molecular weight and lactide to glycolide copolymer ratio used the deterioration time of polymer may vary from several months to several years.

For instance, nanoparticles of the invention are functionalized with PLGA, PLA or PGA, having a molecular weight ranging from about 4000 Da to about 115000 Da

For instance, nanoparticles of the invention are functionalized with PLGA, PLA or PGA, having a molecular weight ranging from about 4000 Da to about 75000 Da.

For instance, nanoparticles of the invention are functionalized with PLGA, PLA or PGA, having a molecular weight ranging from about 4000 Da to about 38000 Da.

For instance, nanoparticles of the invention are functionalized with PLGA having a molecular weight ranging from about 4000 Da to about 17000 Da

In particular, nanoparticles of the invention are functionalized with PLGA having a molecular weight ranging from about 7000 Da to about 17000 Da.

As an exemplary embodiment, the nanoparticles are functionalized with PLGA-Resomer™-RG502H (Sigma-Aldrich).

In some embodiments, nanoparticles of the invention can be associated with one or more types of polymers (e.g. a plurality of polymers) that may optionally further include functional groups. In particular, the polymer can be a synthetic or natural polymer, or a combination of these.

According to some embodiments, the NPs of the invention may comprise, or be functionalized, by one or more sets of polymeric material.

For instance, the additional polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates.

Preferably, the additional polymeric material, when present, may consist of one or more other type of polyester.

Besides poly(lactic acid-co-glycolic acid) (PLGA), poly(lactic acid) (PLA), poly(glutamic acid) (PGA), polycaprolactone (PCL), or other polyester, non-limiting examples of specific polymers which can be functionalized to the nanoparticles include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), and trimethylene carbonate, polyvinylpyrrolidone.

According to one particular embodiment, the nanoparticle is functionalized by polyethylene-glycol (PEG).

According to one particular embodiment, the nanoparticle is not functionalized by polyethylene-glycol (PEG).

Methods for the functionalization of nanoparticles are known in the Art. Accordingly, reference is made to Perrier et al. («Methods for the Functionalisation of Nanoparticles: New Insights and Perspectives»; 2010; Chem. Eur. J. 2010, 16, 11516-11529). In a non-limitative manner, such functional groups may comprise or consist of one or more functional groups selected from: alkyl, alkenyl, alkynyl, phenyl, halo, fluoro, chloro, bromo, iodo, hydroxyl, carbonyl, aldehyde, haloformyl, carbonate ester, carboxylate, ester, methoxy, hydroperoxy, peroxy, ether, hemiacetal, hemiketal, acetal, ketal, orthoester, methylenedioxy, orthocarbonate ester, carboxalide, amine, imine, imide, azide, azo(diimide), cyanate, isocyanate, nitrate, nitrile, isonitrile, nitrosooxy, nitro, nitroso, oxime, pyridyl, sulfhydryl, sulfide, disulfide, sulfinyl, sulfonyl, sulfino, sulfothiocyanate, isothiocyanate, thiol, carbonothioyl, phosphino, phosphono, phosphate, borono, boronate, borino, and borinate functional groups.

In particular, such a nanoparticle may be an amine-modified nanoparticle.

In particular, such a nanoparticle may be a nanoparticle bound to (i.e. functionalized by) at least one additional compound, such as a fluorophore.

According to one embodiment, the nanoparticle is suitable for administration, or co-administration with an anthracycline, or any other cardiomyopathy-inducing drug.

In a general manner, the nanoparticle is meant to modulate, or even inhibit, the effects of a cardiomyopathy-inducing drug (i.e. anthracycline) or the consequences of a cardiomyopathy. Hence, while administration, or co-administration with such drugs may be considered in certain embodiments, it will be readily understood that such nanoparticles according to the invention preferably do not encapsulate any cardiomyopathy-inducing drug and/or an anthracycline and/or an amino-4-quinoleine.

Preferably, nanoparticles according to the invention do not encapsulate any active agent; in particular, they do not encapsulate any cardiomyopathy-inducing drug, anthracycline and/or an amino-4-quinoleine.

Also preferably, the nanoparticle is not covalently bound to a cardiomyopathy-inducing drug and/or an anthracycline and/or an amino-4-quinoleine.

Most preferably, a nanoparticle according to the invention thus does not encapsulate, nor is covalently bound to, an anthracycline or an amino-4-quinoleine.

According to some alternative embodiments, the nanoparticle is covalently or non-covalently bound to one or more targeting moieties. For example, as is more fully outlined below, the nanoparticles of the invention may be covalently or non-covalently bound to a targeting moiety to target the nanoparticles (including bioaffecting agents associated with the nanoparticles) to a specific cell type such as tumor cells or cardiomyocytes. Similarly, a targeting moiety may include components useful in targeting the nanoparticles to a particular subcellular location. As will be appreciated by those in the art, the localization of proteins within a cell is a simple method for increasing effective concentration. For example, shuttling a drug into the nucleus confines them to a smaller space thereby increasing concentration. The physiological target may simply be localized to a specific compartment, and the agent must be localized appropriately. More than one targeting moiety can be conjugated or otherwise associated with each nanoparticle, and the target molecule for each targeting moiety can be the same or different.

However, it will be readily understood herein that nanoparticles of the invention may optionally comprise one or more targeting moieties.

The nanoparticles of the subject invention are also useful as biomedical devices (i.e. plastics). Exemplary applications of the nanoparticles include formation into shunts, cannulas, dressings, endotracheal tubes, percutaneous devices, intra-ocular lenses, contact lenses, sutures, screws, patches and any desired other implants that can be made of plastics.

Hence, the invention further relates to nanoparticles of the invention for the preparation of such biomedical devices.

Pharmaceutical Compositions & Kits

Herein is described a pharmaceutical composition comprising a biocompatible and biodegradable poly(lactic-co-glycolic acid) (PLGA) nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle.

In particular, herein is described a pharmaceutical composition comprising a biocompatible and biodegradable poly(lactic-co-glycolic acid) (PLGA) nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle; for use as a medicament.

Herein is also described a pharmaceutical composition comprising a biocompatible and biodegradable poly(lactic-co-glycolic acid) (PLGA) nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle; and at least one non-encapsulated anthracycline, in particular an anthracycline selected from a group consisting of: daunorubicin, doxorubicin, epirubicin, farmorubicin, idarubicin, mitoxantrone, pixantrone; and their pharmaceutically acceptable salts.

According to another main embodiment, the invention relates to a kit comprising:
a first part comprising a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle; and
a second part comprising an anthracycline, in particular an anthracycline selected from a group consisting of: daunorubicin, doxorubicin, epirubicin, farmorubicin, idarubicin, mitoxantrone, pixantrone; and their pharmaceutically acceptable salts.

In particular, the kit may comprise:
a first part comprising a biocompatible and biodegradable nanoparticle having a diameter of 50 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle; and
a second part comprising an anthracycline, in particular an anthracycline selected from a group consisting of: daunorubicin, doxorubicin, epirubicin, farmorubicin, idarubicin, mitoxantrone, pixantrone; and their pharmaceutically acceptable salts.

In particular, the kit may comprise:
a first part comprising a biocompatible and biodegradable nanoparticle having a diameter of 50 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle; and
a second part comprising doxorubicin, or one of its pharmaceutically acceptable salts.

Herein is also described a pharmaceutical composition comprising (i) a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle, and (ii) at least one anthracycline that is not encapsulated within the nanoparticle, nor covalently bound to the nanoparticle.

Herein is also described a use of a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle; for the preparation of a pharmaceutical composition, and in particular for the preparation of a pharmaceutical aerosol composition.

Herein is also described a therapeutic method, comprising administering to patient in need thereof a pharmaceutical aerosol composition comprising a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle.

According to particular embodiments, the nanoparticles according to the invention, or pharmaceutical compositions thereof, are suitable for any route of administration, which may thus include any enteral or parenteral or topical route of administration.

More particularly, the nanoparticles according to the invention, or pharmaceutical compositions thereof, are suitable for oral, nasal, tracheal, pulmonary intraperitoneal, intravenous, intraarterial or intrapericardial administration.

According to exemplary embodiments, the nanoparticles according to the invention, or pharmaceutical compositions thereof, are in a form which is suitable for intranasal administration, tracheal administration, and/or pulmonary administration.

According to exemplary embodiments, the nanoparticles according to the invention, or pharmaceutical compositions thereof, may thus be provided and/or administered in the form of a pharmaceutical aerosol composition, or alternatively in a form which is compatible with the formation of an aerosol, such as a suspension aerosol formulation.

According to one exemplary embodiment, the nanoparticles according to the invention are thus provided in the form of a pharmaceutical aerosol composition comprising a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle.

Such pharmaceutical aerosol compositions may be generated by an aerosol generating device, from an aerosol formulation comprising the nanoparticles according to the invention.

An aerosol formulation generally comprises two components: the product concentrate (in this case the nanoparticle(s) or a composition thereof) and the propellant. The product concentrate is the active drug combined with additional ingredients or co-solvents required to make a stable and efficacious product. The concentrate can be a solution, suspension, emulsion, semisolid, or powder. The propellant provides the force that expels the product concentrate (i.e. from a container) and additionally is responsible for the delivery of the formulation in the proper form (i.e., spray, foam, semisolid). When the propellant is a liquefied gas or a mixture of liquefied gases, it can also serve as the solvent or vehicle for the product concentrate. If the product characteristics are to change on dispensing, additional energy in the form of a mechanical breakup system may be required. Such formulations are well known in the Art.

Nanoparticles intended for intranasal and pulmonary delivery (systemic and local) can be administered as aqueous solutions or suspensions, as solutions or suspensions in halogenated hydrocarbon propellants (pressurized metered-dose inhalers), or as dry powders. Metered-dose spray pumps for aqueous formulations, pMDIs, and DPIs for nasal delivery, are available from, for example, Valois of America or Pfeiffer of America.

Aqueous formulations must generally be aerosolized by liquid nebulizers employing either hydraulic or ultrasonic atomization, propellant-based systems require suitable pressurized metered-dose inhalers (pMDIs), and dry powders require dry powder inhaler devices (DPIs) which are capable of dispersing the drug substance effectively. For aqueous and other non-pressurized liquid systems, a variety of nebulizers (including small volume nebulizers) are available to aerosolize the formulations. Compressor-driven nebulizers incorporate jet technology and use compressed air to generate the liquid aerosol.

The nanoparticulate dispersions used in making aqueous aerosol compositions can be made by wet milling or by precipitation methods known in the art. Dry powders containing drug nanoparticles can be made by spray drying or freeze-drying aqueous dispersions of drag nanoparticles. The dispersions used in these systems may or may not contain dissolved diluent material prior to drying. Additionally, both pressurized and non-pressurized milling operations can be employed to make nanoparticulate drug compositions in non-aqueous systems.

In a non-aqueous, non-pressurized milling system, a non-aqueous liquid which has a vapor pressure of 1 atm or less at room temperature is used as a milling medium and may be evaporated to yield dry nanoparticulate drag and surface modifier. The non-aqueous liquid may be, for example, a high-boiling halogenated hydrocarbon. The dry nanoparticulate drag composition thus produced may then be mixed with a suitable propellant or propellants and used in a conventional pressurized metered-dose inhalers (pMDI).

When the formulations are propellant-based systems, they may comprise the nanoparticles according to the invention and a surface modifier.

Such formulations may be prepared by wet milling the coarse drug substance and surface modifier in liquid propellant, either at ambient pressure or under high pressure conditions. Alternatively, dry powders containing drug nanoparticles may be prepared by spray-drying or freeze-drying aqueous dispersions of drug nanoparticles and the resultant powders dispersed into suitable propellants for use in conventional pMDIs. Such nanoparticulate pMDI formulations can be used for either nasal or pulmonary delivery. For pulmonary administration, such formulations afford increased delivery to the deep lung regions. Concentrated aerosol formulations can also be employed in pMDIs.

Aerosol formulations comprising the nanoparticles of the invention may advantageously be prepared and/or administered using a nebulizer, which may thus include pneumatic nebulizers (i.e. jet nebulizers), mechanical nebulizers (i.e. soft mist inhalers) and electrical nebulizers (i.e. ultrasonic wave nebulizers or Vibrating Mesh Technology (VMT) nebulizers).

According to one exemplary embodiment, the invention relates to an aerosol pharmaceutical composition comprising a biocompatible and biodegradable nanoparticles having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and/or a polyester nanoparticle.

According to one exemplary embodiment, the invention relates to an aerosol generating device comprising a pharmaceutical composition comprising biocompatible and biodegradable nanoparticles having a diameter of 100 nm or less, wherein the nanoparticles are selected from: poly(lactic-co-glycolic acid) (PLGA) nanoparticles, poly(lactic acid) (PLA) nanoparticles, poly(glutamic acid) (PGA) nanoparticles, polycaprolactone (PCL) nanoparticles, and/or polyester nanoparticles

According to one exemplary embodiment, the invention relates to an aerosol generating device comprising:
means for generating an aerosol;
a storage container;
a pharmaceutical composition comprising biocompatible and biodegradable nanoparticles having a diameter of 100 nm or less, wherein the nanoparticles are selected from: poly(lactic-co-glycolic acid) (PLGA) nanoparticles, poly(lactic acid) (PLA) nanoparticles, poly(glutamic acid) (PGA) nanoparticles, polycaprolactone (PCL) nanoparticles, and/or polyester nanoparticles.

According to some embodiments, the aerosol generating device is a propellant-based aerosol generating device.

According to some alternative embodiments, the aerosol generating device is a propellant-free aerosol generating device.

According to exemplary embodiments, the aerosol generating device is a nebulizer apparatus.

In particular, the invention related to a nebulizer apparatus comprising a liquid pharmaceutical composition comprising biocompatible and biodegradable nanoparticles having a diameter of 100 nm or less, wherein the nanoparticle is selected from: poly(lactic-co-glycolic acid) (PLGA) nanoparticles, poly(lactic acid) (PLA) nanoparticles, poly(glutamic acid) (PGA) nanoparticles, polycaprolactone (PCL) nanoparticles, and/or polyester nanoparticles.

In particular, the invention relates to a nebulizer apparatus comprising:
means for generating an aerosol;
a storage container;
a liquid pharmaceutical composition comprising biocompatible and biodegradable nanoparticles having a diameter of 100 nm or less, wherein the nanoparticle is selected from: poly(lactic-co-glycolic acid) (PLGA) nanoparticles, poly(lactic acid) (PLA) nanoparticles, poly(glutamic acid) (PGA) nanoparticles, polycaprolactone (PCL) nanoparticles, and/or polyester nanoparticles.

Hence, methods for making a pharmaceutical aerosol composition are further reported herein.

Accordingly, the invention further relates to a method for forming an aerosol, in particular for the preparation of a pharmaceutical aerosol composition, comprising the steps of:
a) providing a liquid pharmaceutical composition comprising biocompatible and biodegradable nanoparticles having a diameter of 100 nm or less, wherein the nanoparticle is selected from: poly(lactic-co-glycolic acid) (PLGA) nanoparticles, poly(lactic acid) (PLA) nanoparticles, poly(glutamic acid) (PGA) nanoparticles, polycaprolactone (PCL) nanoparticles, and/or polyester nanoparticles;
b) nebulizing said composition so as to form an aerosol.

The present disclosure is further illustrated by the following examples.

### EXAMPLES

Material & Methods

Materials

Acetonitrile, 3-aminopropyltriethoxysilane (APTES), poly(lactic-co-glycolic acid) (PLGA-Resomer™-RG502 H), 1-ethyl-3-(3-diméthylaminopropyl)carbodiimide (EDC), dimethylformamide (DMF), and chemicals (Bafilomycin A1, chloroquine, doxorubicin) were purchased from Sigma Aldrich (St Quentin Fallavier, France). N-hydroxysuccinimide (NHS) was obtained from Fluka.

Particles Preparation and Characterization

SiO₂-NPs Functionalization with PLGA.

Silica nanoparticles (SiO 2-NPs) of about 21 nm diameter incorporating or not fluorescein isothiocyanate (FITC) as a fluorophore, have shown an emission spectra at 520 nm under 495 nm excitation. SiO₂—NPs were functionalized with PLGA following two steps. First, APTES was grafted at the SiO₂—NPs surface in order to create hooks, and then, PLGA was reacted with previously grafted APTES. Briefly, 50 mg of SiO₂—NPs were suspended by sonication in 15 mL of acetonitrile. 250 μL of APTES were added dropwise, and the suspension was agitated at 50° C. for 24 h. The NPs were recovered by centrifugation (4000 g for 10 min), washed three times in ethanol and dried under vacuum. Then, 30 mg amine-modified nanoparticles were suspended in 15 mL of DMF. In parallel, 73 mg of PLGA were dissolved in 15 ml of DMF and EDC and NHS were added to the PLGA solution. Then, the PLGA solution was added to the NPs suspension which was agitated at 40° C. for 2 h. The NPs were recovered by centrifugation (4000 g for 10 min), washed three times with distilled water and dried under vacuum.

SiO₂-MPs Functionalization with PLGA.

Silica microparticles (SiO₂-MPs) of about 200 nm, incorporating or not FITC, were functionalized with PLGA using the same procedure as described for SiO₂-NPs following the protocol already described (Rahman et al. 2006). FITC has been incorporated in those SiO₂-MPs exactly in the same proportion as for SiO₂-NPs.

Instrumentation.

Particle shape and size were examined via transmission electron microscopy (TEM), using a Philips Model CM20 microscope (Eindhoven, The Netherlands) for SiO₂-NPs and a scanning electron microscopy (SEM) using a JEOL JSM-6510LV for SiO₂-MPs. Size distribution was assayed by measuring the size of 100 particles using Image J software. Zeta potential measurements were analyzed by dynamic light scattering (DLS) using a Zetasizer Nano (Malvern instruments, Malvern, UK). The luminescence of samples in suspension at a water concentration of 1 mg/mL was studied with a Jobin-Yvon Model Fluorolog FL3-22 spectrometer (HORIBA Scientific, Montpellier, France) equipped with a R928 Hamamatsu photomultiplier and a 450-W Xe excitation lamp.

Cell Culture

Rat H9C2 cardiomyoblasts (American Type Culture Collection, Rockville, U.S.A.) were grown in DMEM media containing 10% heat-inactivated FBS under 5% CO2 and 95% air at 37° C. H9C2 serve as an animal-free alternative sharing many physiological properties of primary cardiac cells. For neonatal rat ventricular myocytes (NRVMs), rats of 1-2 days old were euthanized and hearts were excised before the atria were removed. Primary culture of NRVMs was subsequently performed as previously described (Manzella et al. 2018; Aging Cell 17(5):e12811). NRVMs were grown in Ham-F12 medium containing 10% heat-inactivated FBS and 10% heat-inactivated HS under 5% CO2 and 95% air at 37° C. Plasmid transfections with GFP-LC3, GFP-RFP-LC3 or RFP-LAMP1 constructs (Addgene) were performed using Lipofectamine 2000 reagent (Life Technologies) in Opti-MEM™ transfection medium (Gibco). NRVMs were transduced with an adenovirus expressing rat MAO-A under the control of the CMV promoter to drive expression of MAO-A. 24 h later, the medium was replaced with and MAO substrate tyramine (500 μM) was applied for 8 h to generate oxidative stress (Santin et al. 2016; Antioxid Redox Signal 25(1):10-27 doi:10.1089/ars.2015.6522).

Cell Death

For quantitative assessment of cardiomyocyte necrosis, LDH release in culture medium was measured using the LDH cytotoxicity assay kit according to the manufacturer's instructions (Biovision). Apoptosis was measured in NRVMs by caspase-3 activation using a commercial kit (Biotium) according to the manufacturer's instructions.

Intracellular Uptake Rate (FACS)

After exposure to NPs-PLGA, cells were trypsinized, thoroughly rinsed in PBS and analyzed with a FACSVerse flow cytometer (BD Biosciences). Minimum of 30,000 cells were analyzed after exclusion of the cellular debris. FACSuite software was used for data acquisition.

Intracellular Localization of NPs-PLGA and MPs-PLGA

Intracellular localization of particles was performed in RFP-LAMP1-transduced cells. Image acquisition was performed with an LSM780 laser scanning confocal microscope (Carl Zeiss).

Autophagosomes/Autolysosomes Visualization

Autophagosomes were visualized in GFP-LC3-transduced cells. Autophagic flux was evaluated in RFP-GFP-LC3-transduced cells. Representative images were taken with an epifluorescence microscope (DM600 microscope, Leica) and quantification of yellow puncta (autophagosomes) and red puncta (autolysosomes) was performed using Image J software.

Lysosomal pH

Acridine Orange (AO).

Acridine Orange (Sigma Aldrich), a weak base which accumulates in acidic organelles, was used to label the lysosomes. After treatments, cells were incubated with 5 μM AO for 10 min at 37° C. and washed twice with PBS. Red fluorescence (corresponding to acidic vesicles staining) was measured using a Varioskan Flash Multimode Microplate Reader with excitation/emission at 480/630 nm. Representative pictures were taken with an epifluorescence microscope (DM600 microscope, Leica).

Lysosensor.

Lysosomal acidification was measured in H9C2 and NRVMs loaded with 2 μM LysoSensor yellow/blue DND-160 (Invitrogen) for 15 min at 37° C. The LysoSensor dye is a ratiometric probe that produces yellow fluorescence in acidic environments, but changes to blue fluorescence in neutral environments. pH calibration was performed following the protocol established by Diwu et al. (Chem Biol. (1999); 6(7):411-418). Briefly, cells were incubated in MES buffer (5 mMNaCl, 115 mMKCl, 1.3 mM MgSO 4, 25 mM MES) supplemented with 10 μM nigericin and 10 μM monensin, for 10 min, with pH adjusted to a range from 3 to 7. The samples were read in a Varioskan Flash Multimode Microplate Reader with excitation at 360 nm. The ratio of emission 440/540 nm was then calculated for each sample.

Cathepsin D Activity

Cathepsin D activity was measured as an indicator of lysosomal proteolytic activity. Enzyme activity was determined with cathepsin D activity fluorometric assay kit according to the manufacturer's protocol (abcam). Fluorescence was then measured with a Varioskan Flash Multimode Microplate Reader at 328/460 nm (excitation/emission).

Western Blots

H9C2 or NRVMs extracts were prepared by lysing in RIPA buffer (50 mM Tris pH 7.2, 500 mM NaCl, 1% Triton X-100, 1 mM EDTA, 100 mM sodium fluoride, 5 mM sodium metavanadate, 10 mM sodium pyrophosphate) and equal protein amounts were subjected to sodium dodecyl sulfate-polyacrylamide gel electrophoresis (SDS-PAGE) in 10-15% gels depending upon protein molecular weight. After electrophoresis, proteins were transferred to nitrocellulose membranes and incubated with the following antibodies: anti-GAPDH, anti-LC3B, anti-cleaved caspase 3, anti-p-p53 from Cell Signaling Technology, anti-p62 from Abnova, anti-Lamp1, anti-cathepsin D from Abcam. Proteins were detected by chemiluminescence with a Bio-Rad ChemiDoc XRS camera. Relative densities were quantified using the ImageLab 5.2.1 software (Bio-Rad). All data were normalized by internal controls.

Cellular and Mitochondrial ROS

Cellular ROS were measured using the fluorescent probe DCFDA assay at a concentration of 5 μM (ThermoFisher Scientific). Fluorescence was quantified using a fluorimeter TECAN infinite proF200 with excitation/emission at 500/530 nm. Mitochondrial ROS were measured by mitoSOX probe (Invitrogen, Molecular Probes). Briefly, the cells were loaded with mitoSOX probe at a final concentration of 5 μM and incubated for 30 min after the indicated treatments. Cells were resuspended in HBSS before reading in Tecan plate reader.

Mitochondrial Dysfunction

The mitochondrial membrane potential was evaluated using JC-1 probe (ThermoFisher Scientific). Before the end of treatments, cells were loaded with JC-1 probe at a concentration of 5 μg/ml for 10 min at 37° C. Then, medium was replaced by HBSS and the fluorescence was recorded at 535 nm (excitation wavelength, emission at 590 nm) to measure the formation of red aggregates and at 485 (excitation wavelength, emission at 530 nm) to measure the presence of green monomers, using a fluorimeter TECAN infinite pro F200. Representative images were taken with an epifluorescence microscope (DM600 microscope, Leica).

Statistical Analysis

Statistical analysis was carried out using Student's t-test or 2-way ANOVA with the Tukey post hoc test, when appropriate. The results are shown as the mean±SEM. Values of p<0.05 were considered to be significant.

In Vivo Experiments

All animal procedures were performed in accordance with International Guidelines on Animal Experimentation and with a French Ministry of Agriculture license. Moreover, this investigation conformed to the guide for Care and Use of Laboratory Animals published by the Directive 2010/63/EU of the European Parliament. All mice were housed in temperature-controlled cages with a 12-h light-dark cycle and given free access to water and food.

Nanoparticle administration: for intratracheal nebulization, animals were anesthetized by intrapulmonary (IP) injection of xylazine/ketamine and were secured to a tray in the supine position. Using a 20 G angiocath, animals were intubated. The board was tilted at 45 degrees and the IA-1C Microsprayer tip (PennCentury, Wyndmoor, PA, USA) was inserted through the lumen of the angiocath. 50 μL of NPs-PLGA were administered via IT delivery and the tip was removed. The animals were then extubated and returned to their cages. At the end of the experimentation, mice were sacrificed and tissues were collected.

Doxorubicin treatment: Mice were injected via tail vein with doxorubicin (5 mg/kg) or normal saline.

Immunofluorescence on Heart Sections

Heart tissues were embedded in optimal cutting temperature compound (OCT) (Sigma-Aldrich) under ice-cold 2-methylbutane. Frozen sections (5 μm) were fixed in 4% paraformaldehyde, followed by permeabilization and blocking in PBS with 0.02% FBS, 1% bovine serum albumin, and 0.3% Triton X-100 at RT. Sections were immunostained overnight with the following antibodies: anti alpha-actinin (GeneTex 103219) followed by secondary Alexafluor antibodies (Molecular Probes). Nuclei were visualized with DAPI. Images were acquired by confocal Microscope Zeiss LSM 780 and ZEN image analysis software (Zeiss).

### Example 1

Characterization of PLGA Functionalized Silica Nanoparticles (NPs-PLGA)

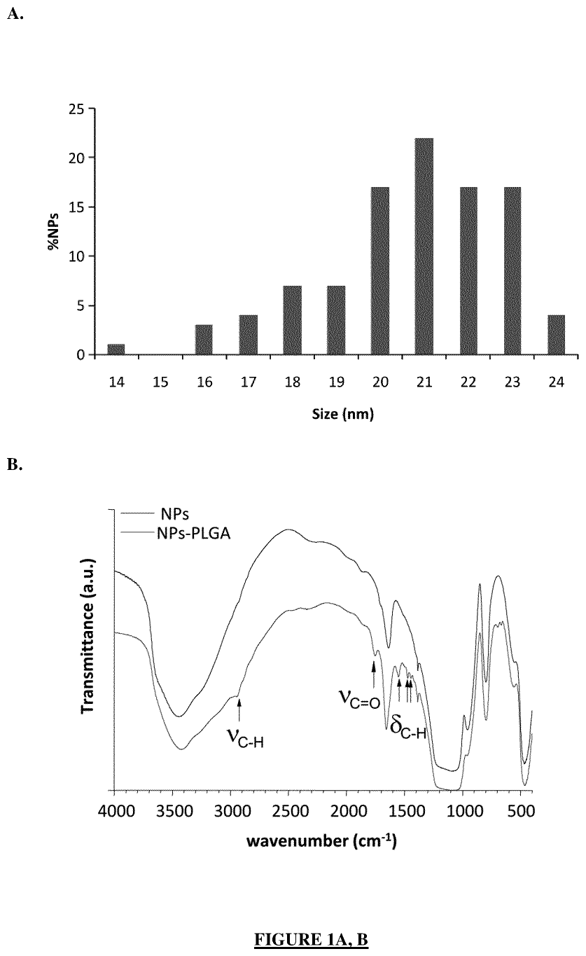

Silica nanoparticles (NPs) were visualized by TEM and appeared spherical and mono-dispersed with an average diameter of 21.3 nm (±2.0 nm) (FIG. 1A). Fluorescein content was optimized in order to maximize the brilliancy of NPs and not change significantly the particle average size (26 nm±2.0 nm). When too diluted, the brilliancy was weaker due to the small number of encapsulated chromophores, but embedding too many fluorophore molecules led to a drastic decrease in brilliancy, caused by confinement and the self-quenching effect. Photoluminescence properties of both NPs and NPs-PLGA were studied in water at a concentration of 1 mg/mL. The photo-luminescence (PL) spectra showed a broad excitation band centered at 495 nm and a broad emission band at 520 nm. Infrared spectra of NPs and NPs-PLGA showed well known Si—O—Si (1070 and 795 cm⁻¹), Si—O (461 cm⁻¹) and (Si—)O—H (3422 cm⁻¹) bands (FIG. 1B). Strong peak at 1630 cm⁻¹ is due to water molecules (FIG. 1B). Additional bands observed on NPs-PLGA spectrum can be attributed to v C-H (2945 cm⁻¹) and v C-H (1550, 1473, 1430 cm⁻¹) of both APTES and PLGA grafted molecules and to v C═O of PLGA (1750 cm⁻¹) (FIG. 1B). The zeta potential for naked NPs was strongly negative: −20.9 mV and −25.6 mV (respectively without and with FITC) whereas when functionalized by PLGA, it became positive at: +5 mV and +10 mV (respectively without and with FITC) showing clearly that PLGA molecule is effectively grafted on the SiO₂ surface.

### Example 2

Cytotoxicity, Intracellular Uptake Rate and Lysosomal Distribution of NPs-PLGA

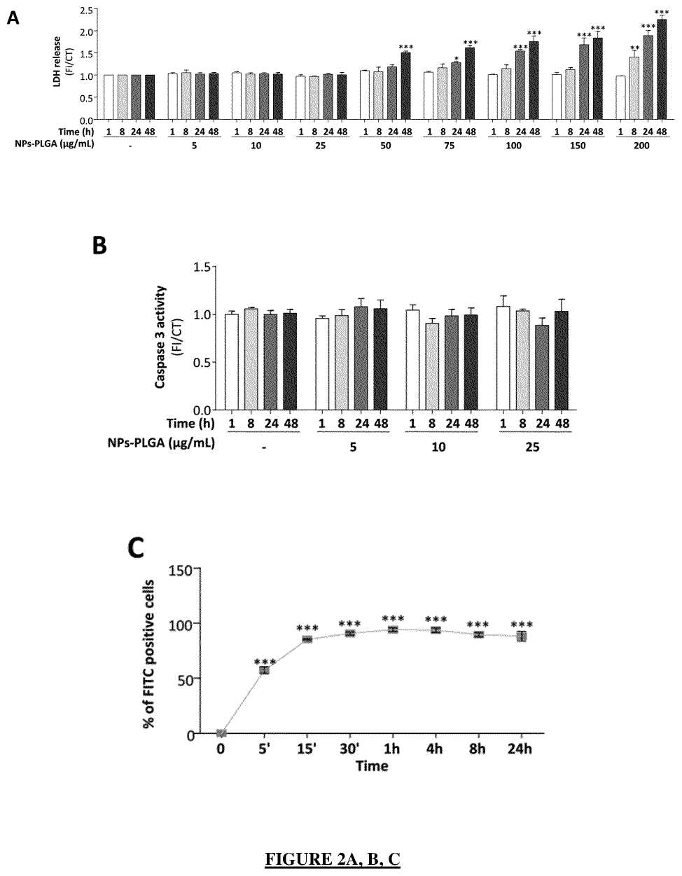

NPs-PLGA were incubated at increasing doses with H9C2 cells in order to determine their intracellular fate and safety. We first analysed the effects of NPs-PLGA on cell necrosis, measured by LDH release, until 48 hours of incubation (FIG. 2A). We found that NPs-PLGA were safe until the dose of 25 μg/mL, while at higher doses (50 to 200 μg/mL), LDH was released in a dose- and time-dependent manner. The absence of induction of apoptosis was verified at doses of 25 μg/mL and lower, by measuring caspase 3 activity (FIG. 2B). We next evaluated the cellular uptake rate of FITC-labelled NPs-PLGA (FITC-NPs-PLGA) by flow cytometry at 25 μg/mL. Within 30 min, 90% of H9C2 cells showed a significant uptake of green fluorescence and this rate remained equal until 24 h of incubation (FIG. 2C). To confirm uptake of the nanoparticles into the lysosomal compartment, confocal imaging was performed using the lysosomal marker construct RFP-LAMP1. Our results confirmed lysosomal addressing of NPs-PLGA at 24 hours, as demonstrated by the strong colocalization between FITC-NPs-PLGA and RFP-LAMP1. Taken together, these data indicate that NPs-PLGA at nontoxic doses are rapidly internalized into H9C2 and trafficked to lysosomes.

### Example 3

NPs-PLGA Mitigate Lysosomal Alkalinisation and Autophagy Dysfunction in H9C2 Treated with Lysosomal Inhibitors.

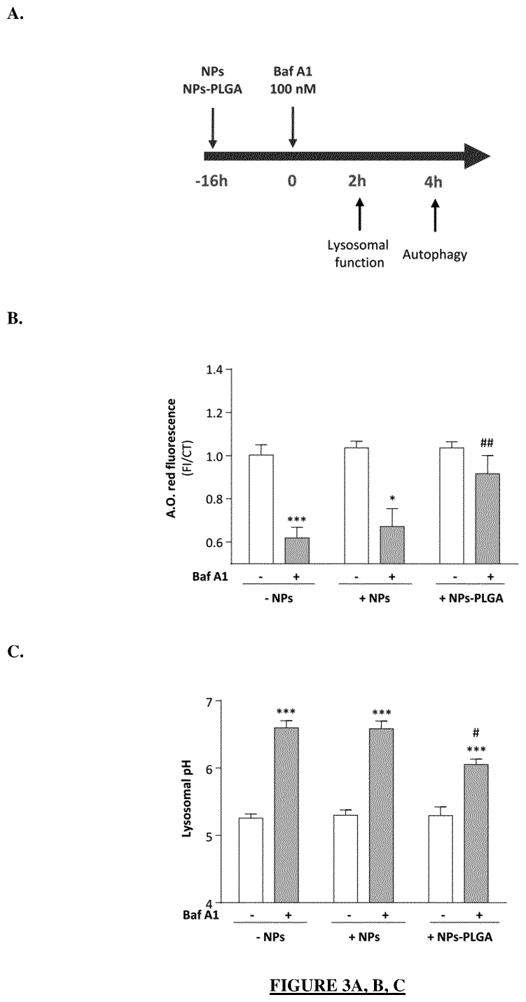

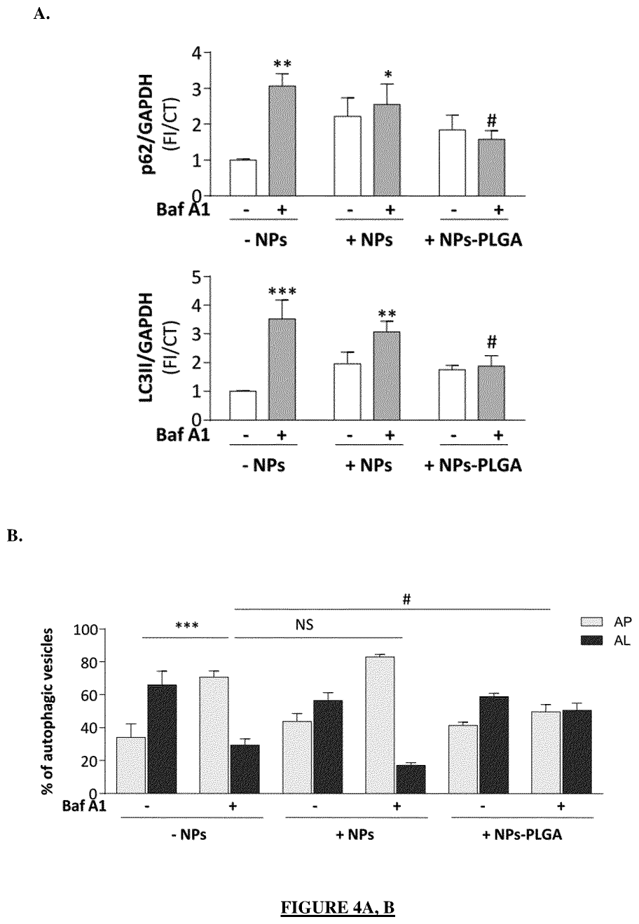

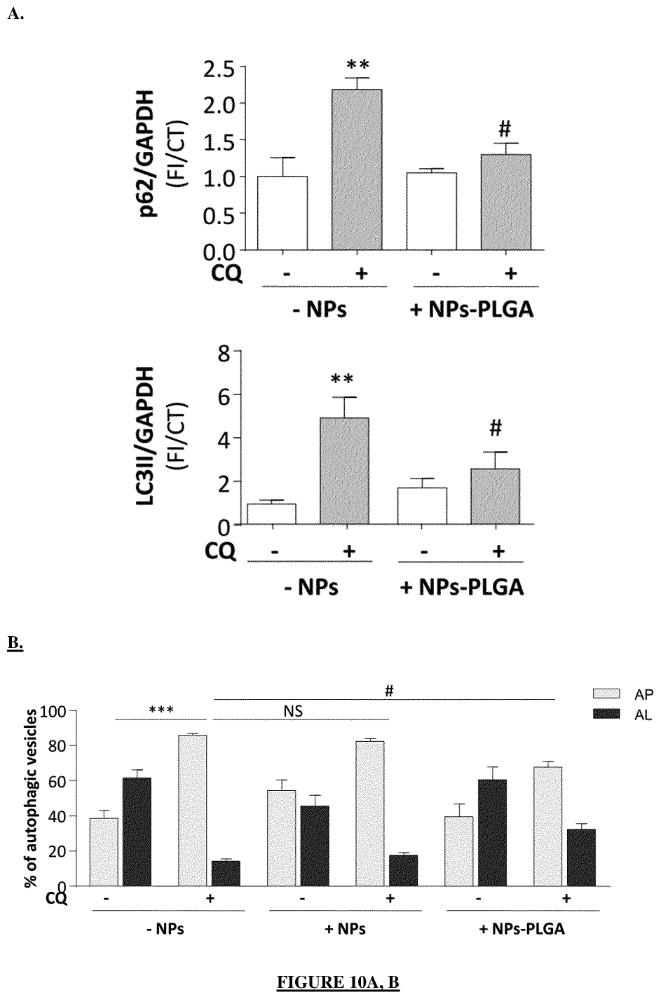

We next sought to investigate whether NPs-PLGA could influence lysosomal acidity in the presence of lysosomotropic agents. We used the potent v-ATPase inhibitor Bafilomycin A1 (Baf A1) to inhibit lysosomal acidification in H9C2 cells, as shown by the loss of Acridine Orange (AO) red puncta (FIG. 3B) and by the rise in lysosomal pH (FIG. 3C). Interestingly, pre-incubation of the cells with NPs-PLGA significantly mitigated lysosomal pH alkalinisation (FIG. 3B-C). As a negative control, the same experiments were performed in the presence of silica nanoparticles devoid of PLGA (NPs). As shown in FIG. 3, these NPs did not prevent lysosomal pH alteration induced by Baf A1, supporting the proper role of PLGA in lysosomal acidification (FIG. 3A-C). Given the beneficial effects of NPs-PLGA on lysosomal pH, we wondered if they could ameliorate autophagic flux in the presence of Baf A1. Immunofluorescence staining showed an accumulation of autophagosomes (AP) in GFP-LC3-transduced H9C2 following Baf A1 treatment, which was confirmed by the increased LC3-II and p62 protein levels (FIG. 4A-B). NPs-PLGA, but not NPs, alleviated the raise of AP markers (FIG. 4A-B). Next, we used a tandem RFP-GFP-LC3 reporter system as an additional monitor of autophagic flux. Consistent with our previous data, Baf A1 led to the accumulation of red+green autophagosomes (AP) and to the reduction of red-only autolysosomes, indicating a defect in autophagosome-lysosome fusion (FIG. 4C). Surprisingly, NPs-PLGA lessened AP accumulation while NPs had no effect (FIG. 4C). These observations were recapitulated in the presence of another lysosomal inhibitor, chloroquine (CQ) (FIG. 10).

### Example 4

NPs-PLGA Prevent Lysosomal Alkalinisation and Autophagy Dysfunction in Doxorubicin-Treated H9C2.

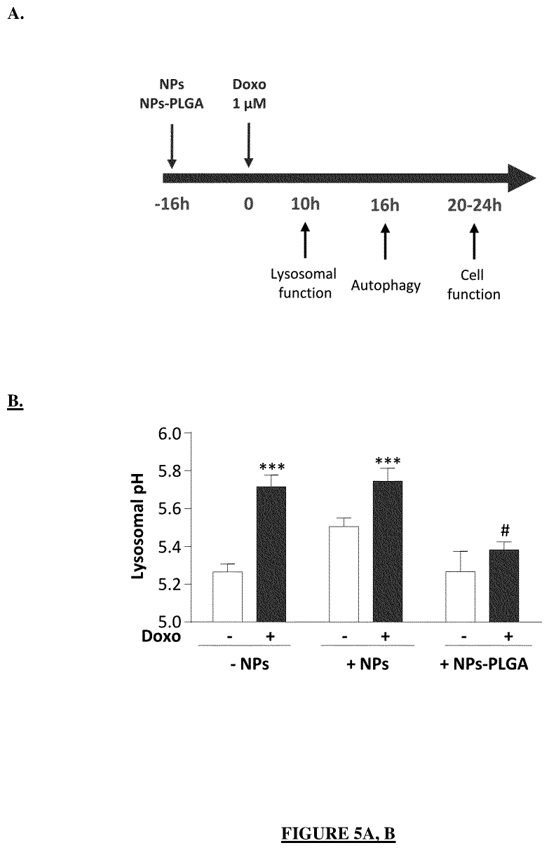

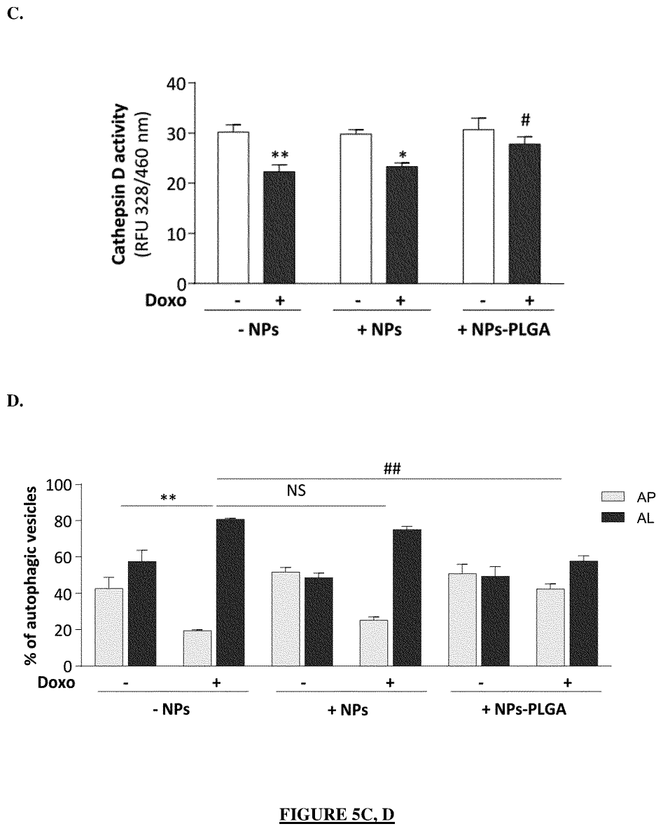

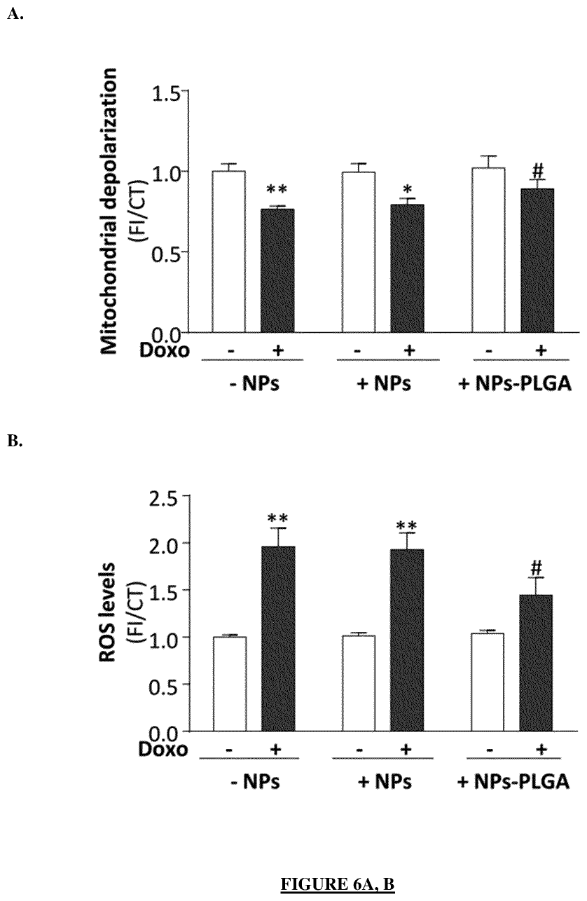

We then studied the effects of NPs-PLGA in a pathological model of lysosomal alteration. The chemotherapeutic drug doxorubicin is known to induce cardiotoxicity and recent studies have demonstrated that doxorubicin adverse effects involved an inhibition of lysosomal acidification in cardiomyocytes (Bartlett et al. (2016; The Biochemical Journal; 473(21):3769-3789) & Li et al. (2016; Circulation; 133(17):1668-1687). We thus investigated the preventive role of NPs-PLGA in doxorubicin-induced lysosomal dysfunction (FIG. 5A). We showed that doxorubicin significantly decreased AO red fluorescence and increased lysosomal pH, as shown with lysosensor blue/yellow ratio (FIG. 5B). NPs-PLGA applied prior to doxorubicin prevented the loss of red fluorescence as well as lysosomal alkalinisation. This effect of NPs-PLGA on lysosomal acidification was accompanied by a preservation of proteosomal activity as shown with cathepsin D activity assay (FIG. 5C). Analysis of the autophagic flux with GFP-RFP-LC3 reporter construct demonstrated that doxorubicin led to an accumulation of autolysosomes (AL), an event that was mitigated with prior application of NPs-PLGA (FIG. 5D). On the other hand, the number of autophagosomes (AP) remained unchanged in the presence of doxorubicin.

### Example 5

Autophagic Flux Improvement Leads to Ameliorated Cell Function and Viability in Doxorubicin-Treated H9C2.

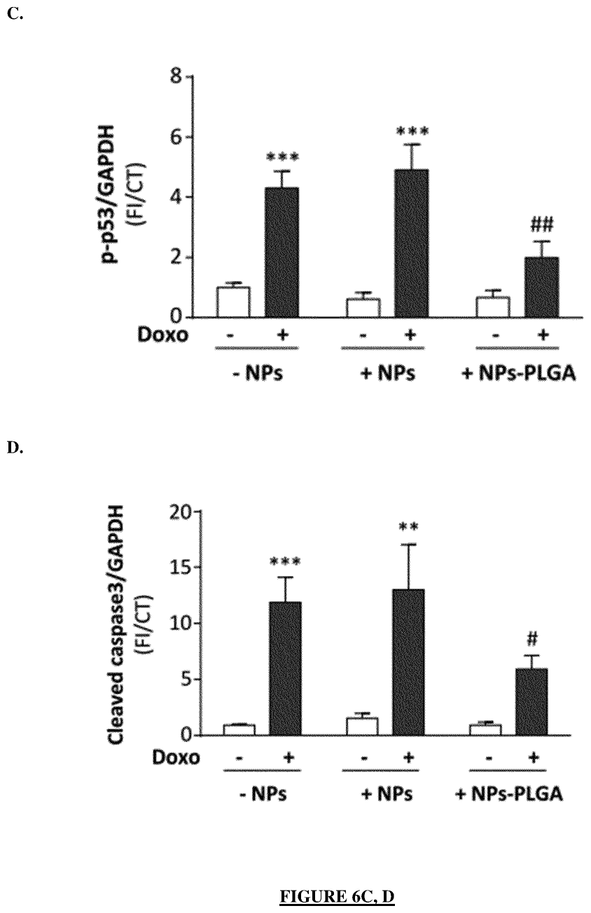

Having evidenced the alleviation of lysosomal impairment and autophagic flux dysfunction in the presence of NPs-PLGA, we next assessed if this could have an impact on the accumulation of damaged mitochondria in the presence of doxorubicin. Mitochondrial dysfunction was evidenced by a drop in mitochondrial membrane potential, measured with JC-1 probe, 10 h after doxorubicin treatment (FIG. 6A). Interestingly, NPs-PLGA but not NPs mitigated this mitochondrial depolarization. Also, an accumulation of oxidative stress 10 h after doxorubicin treatment could be partially reduced with NPs-PLGA but not NPs (FIG. 6B). Finally, NPs-PLGA limited doxorubicin-induced deleterious effects on cell death, as demonstrated by a decreased expression of p-p53 and cleaved caspase3 (FIG. 6C-D). These results highlight the key role of lysosomal dysfunction in the exacerbation of doxorubicin cardiotoxicity. Much interestingly, we show that acting on lysosomes with NPs-PLGA improves the autophagic removal of damaged mitochondria and consequently mitigates anthracycline-linked cellular dysfunction.

### Example 6

NPs-PLGA Improve Lysosomal Function, Autophagic Flux and Cell Viability in Doxorubicin-Treated Neonatal Rat Ventricular Myocytes (NRVMs)

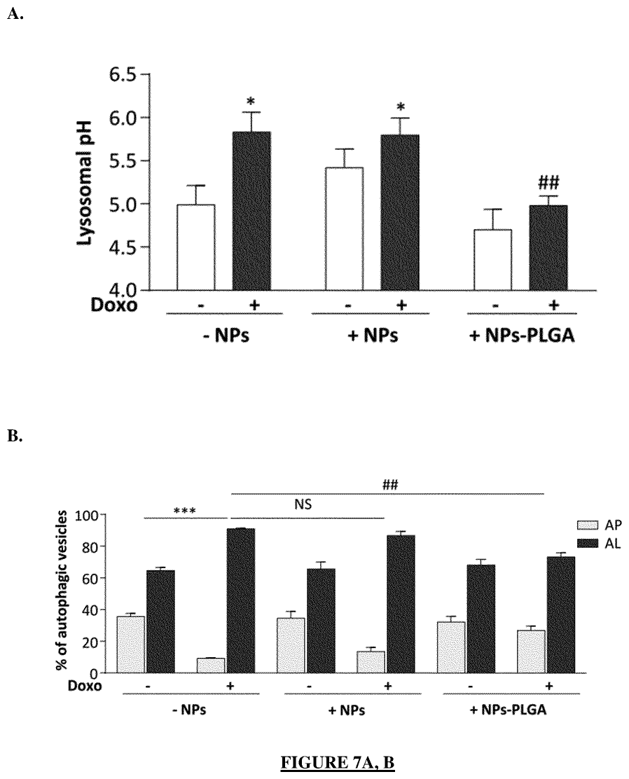

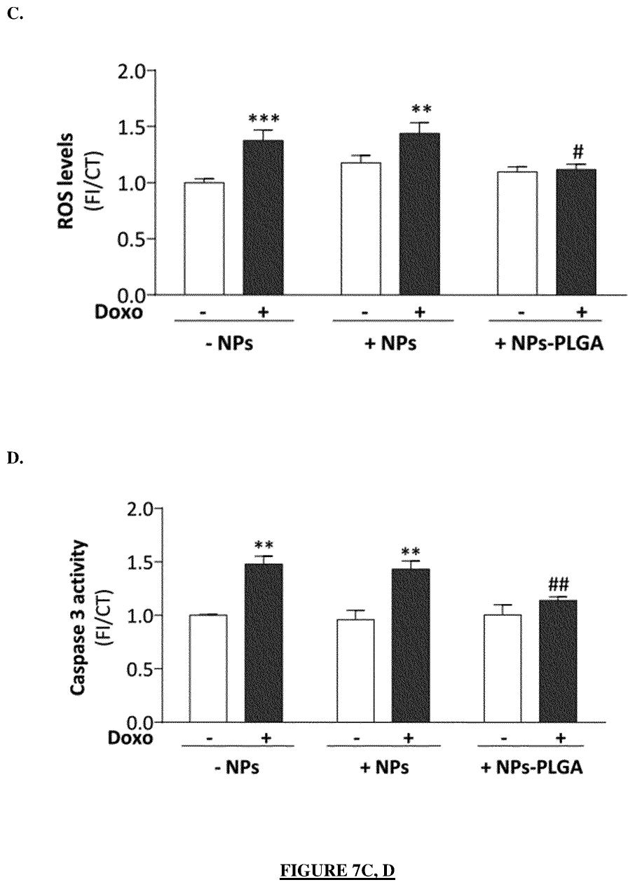

In order to translate our findings in a more physiological model, we used primary cultures of cardiomyocytes. In this model, NPs-PLGA appeared non toxic, even at high doses. However, compared to our previous experiments in H9C2, a much higher dose of NPs-PLGA was required to acidify the lysosomes in the presence of doxorubicin (200 μg/mL) (FIG. 7A). Likewise, with this dose, a prevention in lysosomal pH alkalinisation was also observed in a model of oxidative stress induced by overactivation of the mitochondrial enzyme MAO-A. At this dose, NPs-PLGA labelled with FITC demonstrated rapid internalization in cardiomyocytes and colocalized with the lysosome marker RFP-LAMP1. Interestingly, NPs-PLGA limited the accumulation of autolysosomes in the presence of doxorubicin while the same dose of NPs did not have any effect (FIG. 7B). Then, the protective role of NPs-PLGA was further confirmed since they mitigated doxorubicin-induced oxidative stress (FIG. 7C) as well as cell apoptosis (FIG. 7D) measured by caspase-3 activation.

### Example 7

Curative Effects of NPs-PLGA Subsequent to Doxorubicin on Cardiomyocytes

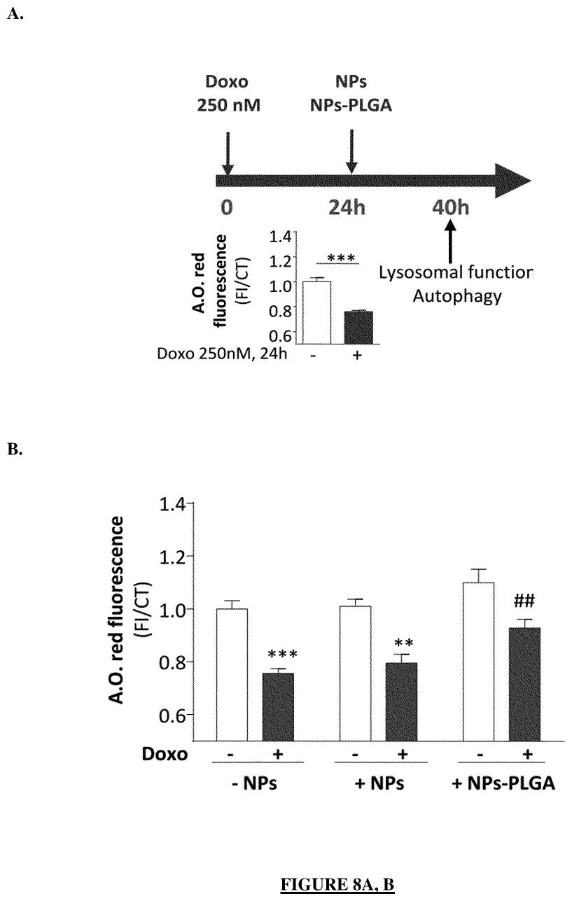

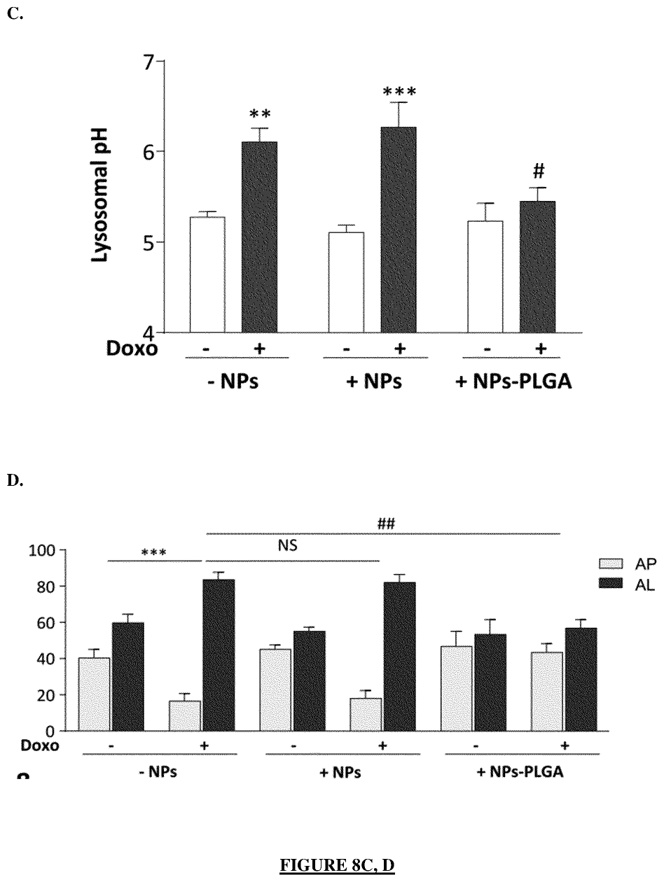

Finally, we evaluated if application of NPs-PLGA post-doxorubicin treatment, at a time where lysosomal dysfunction is already established, could still acidify lysosomes and prevent cellular impairment (FIG. 8A). As the dose of doxorubicin used in prior experiments led to a rapid cell death in only 24 hours, we decided to decrease the concentration of doxo to 250 nM, which allows long term analysis of cell parameters. Doxorubicin used at 250 nM triggered a decrease in A.O. red fluorescence at 24 h (FIG. 8A) and this alteration was maintained until 40 h (FIG. 8B). Of great interest, NPs-PLGA applied 24 h after doxorubicin were able to improve lysosomal pH, meaning that they could reacidify

dysfunctional lysosomes (FIG. 8B-C). In addition, both lysosomal alteration and autophagy dysfunction were improved by NPs-PLGA, while NPs were devoid of beneficial effects (FIG. 8D-E). Finally, doxorubicin-induced mitochondrial ROS increase, as evidenced by mitoSOX staining, was mitigated by NPs-PLGA (FIG. 8F). Altogether, our data provide evidences that acidic nanoparticles can reacidify dysfunctional lysosomes, improve autophagic flux and cell function in the pathological context of doxorubicin-induced cardiomyocyte dysfunction.

### Example 8

Size Effect of NPs-PLGA Vs. MPs-PLGA

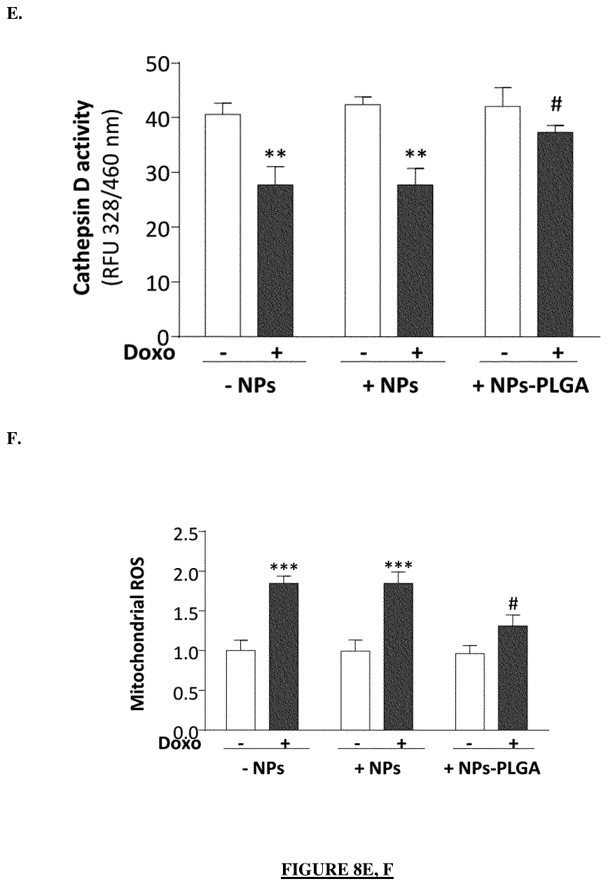

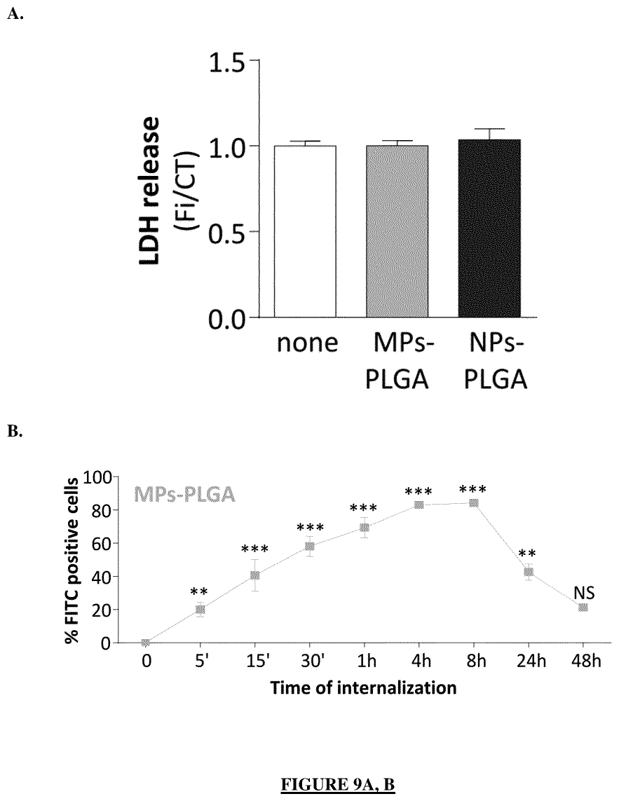

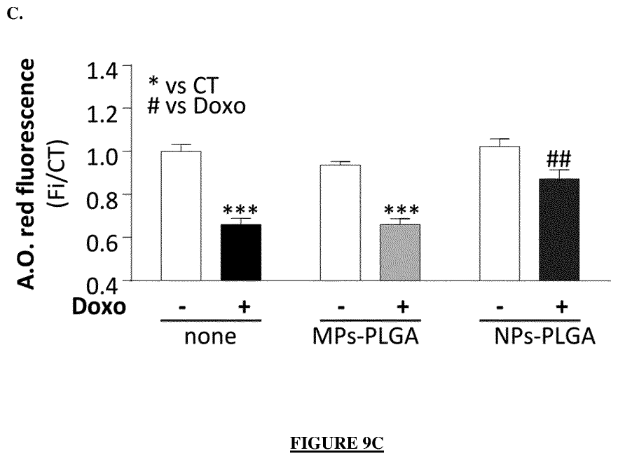

As particles size can greatly affect their cellular internalization and their consequent functional effects, we developed PLGA-grafted silica particles of bigger size (microparticles, MPs-PLGA). MPs were first visualized by Scanning Electron Microscopy (SEM) and appeared agglomerated with an ovoid shape and a poly-dispersed size distribution. Average particle length was around 215 nm (±60 nm) and 138 nm (±30 nm) width for FITC-labelled MPs. MPS without FITC were slightly smaller with average length of 175 nm (±27 nm) and width of 124 nm (±30 nm). Infrared spectra of MPs showed Si—O—Si, Si—O, (Si—)O—H and water molecules bands that were similar to NPs. When incubated with NRVMs, MPs-PLGA were non toxic at 200 μg/mL (FIG. 9A). While MPs-PLGA exhibited a similar internalization rate compared to NPs-PLGA until 8 h of incubation (FIG. 9B), confocal images of LAMP1-transfected NRVMs showed that they were not addressed efficiently to lysosomes. Hence, nanoparticle size appears as a determinant factor for lysosomal addressing. Consequently, MPs-PLGA, contrary to NPs-PLGA, did not induce any improvement in lysosomal pH after doxorubicin treatment (FIG. 9C).

### Example 9

Nanoparticles Administration Via Intratracheal Nebulization on Mice and its Effect on Autophagic Markers LC3-II and p62.

This experiment provides an in vivo proof-of-concept of the prevention of autophagy dysfunction induced by doxorubicin, following intratracheal nebulization of nanoparticles according to the invention in mice.

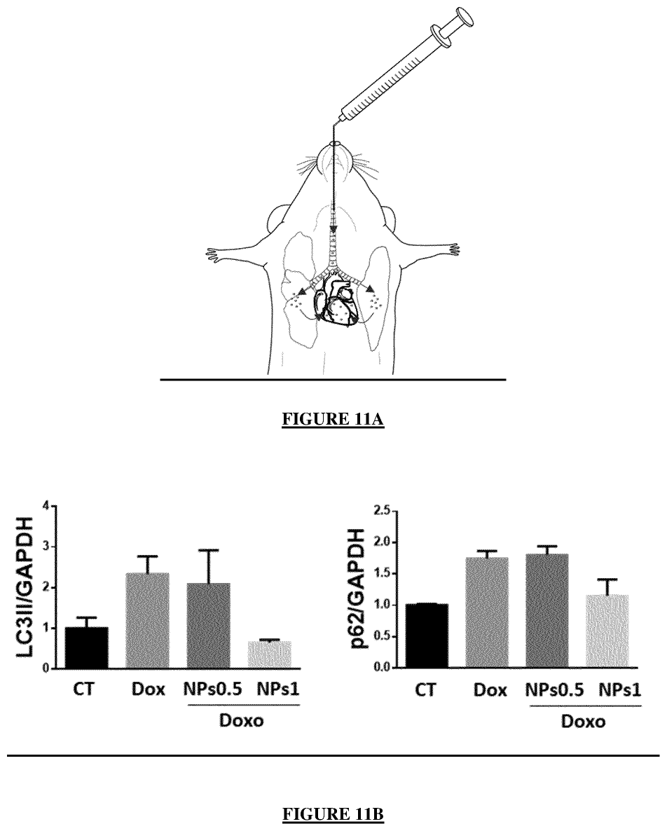

FIG. 11A provides a schematic representation of the administration. After reaching the lungs from the trachea, NPs cross the pulmonary vein and accumulate within the heart. The presence of nanoparticles on cardiac cryosections is verified on mice 1 h after NPs intra-tracheal nebulization by immunofluorescence, based on co-localization of alpha-actinin and labeled nanoparticles with FITC (data not shown).

Then, based on immunoblot signal intensities autophagic markers LC3-II and p62 in cardiac homogenates of mice treated or not with Doxorubicin (5 mg/kg i.v.) and NPs-PLGA (0.5 or 1 mg/kg intratracheal) are compared. The outcome is normalized based on GAPDH signal and provided as FIG. 11B.

While Doxorubicin induces an increase of LC3-II and p62, NPs at 1 mg/kg prevent autophagy dysfunction as shown by the normalization of LC3-II and p62 levels.

### Example 10

Nanoparticles Administration Via Intratracheal Nebulization Prevents Doxorubin-Induced Cardiac Remodeling and Impairment of Cardiac Function In Vivo.

This experiment provides an in vivo proof-of-concept of the prevention of doxorubin-induced cardiac remodeling and impairment of cardiac function, following intratracheal nebulization of nanoparticles according to the invention in mice.

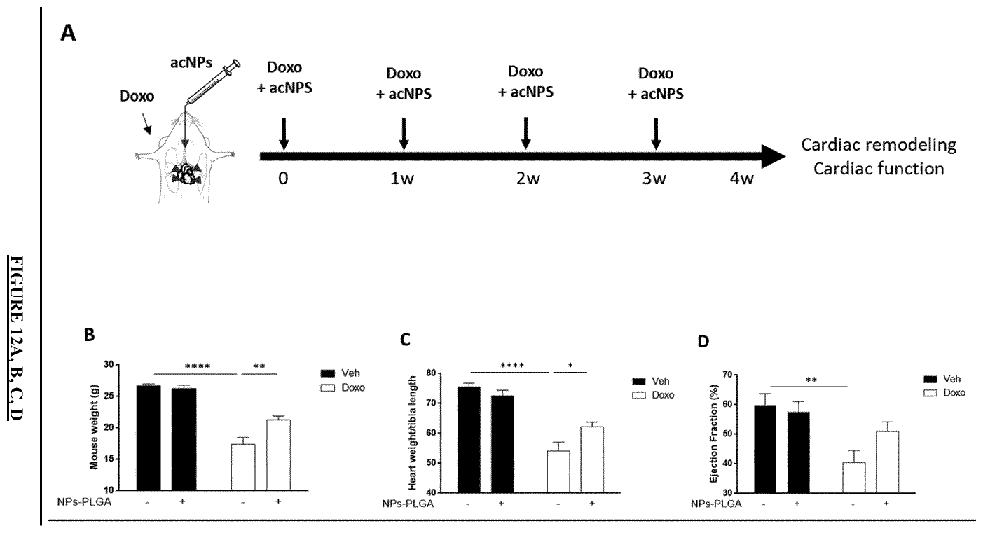

FIG. 12A provides a schematic representation of the administration. Doxorubicin is administered by the intravenous route as in Example 9, followed by intratracheal nebulization of nanoparticles (NPs) according to the invention. The experiment is followed for about four weeks. The outcome of the experiment is determined based on mouse weight variation (12B), the heart weight/tibia length ratio (12C) and ejection fraction (12D) for assessing cardiac function. The vehicle (Veh) refers to the mice for which Doxorubicin was not administered. Each group originally includes nine mice. Asterisks mark differences which are statistically relevant within the doxorubicin-treated groups, in comparison with the vehicle/control groups.

Overall, the in vivo experiment thus demonstrates a lack of toxicity over four weeks for the nanoparticles alone, but also prevention of the risk of doxorubicin-induced cardiac remodeling and impairment of cardiac function.

1. A method for treating or preventing a drug-induced cardiomyopathy comprising a step of administering to a patient in need thereof a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and a polyester nanoparticle.

2. A method for treating or preventing anthracycline-toxicity comprising a step of administering to a patient in need thereof a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and a polyester nanoparticle.

3. (canceled)

4. The method according to claim 1, wherein the drug-induced cardiomyopathy is induced by an anthracycline selected from the group consisting of: daunorubicin, doxorubicin, epirubicin, farmorubicin, idarubicin, mitoxantrone, and pixantrone; and their pharmaceutically acceptable salts.

5. The method according to claim 4, wherein the drug-induced cardiomyopathy is induced by doxorubicin, or a pharmaceutically acceptable salt thereof.

6. The method according to claim 1, wherein the drug-induced cardiomyopathy is induced by an amino-4-quinoleine or a derivative thereof selected from the group consisting of: chloroquine, hydroxychloroquine, and amodiaquine; or a pharmaceutically acceptable salt thereof.

7. The method according to claim 1, wherein the nanoparticle is a silica-based nanoparticle (SiNP).

8. The method according to claim 1, wherein the nanoparticle has a diameter of less than 50 nm.

9. The method according to claim 1, wherein the nanoparticle is not covalently bound to, nor encapsulates, an anthracycline or an amino-4-quinoleine.

10. The method according to claim 1, wherein the nanoparticle is selected from: a poly(lactic-co-glycolic acid) (PLGA) nanoparticle, a poly(lactic acid) (PLA) nanoparticle, and a poly(glutamic acid) (PGA) nanoparticle.

11. The method according to claim 1, wherein the nanoparticle is formulated as a pharmaceutical aerosol composition.

12. A kit comprising:
a first part comprising a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and a polyester nanoparticle; and
a second part comprising at least one anthracycline.

13. A pharmaceutical aerosol composition comprising a biocompatible and biodegradable nanoparticle having a diameter of 100 nm or less, wherein the nanoparticle is selected from: a poly(lactic-co-glycolic acid) (PLGA) nanoparticle, a poly(lactic acid) (PLA) nanoparticle, a poly(glutamic acid) (PGA) nanoparticle, a polycaprolactone (PCL) nanoparticle, and a polyester nanoparticle.

14. (canceled)

15. An aerosol generating device comprising: a pharmaceutical composition comprising biocompatible and biodegradable nanoparticles having a diameter of 100 nm or less, wherein the nanoparticles are selected from: poly(lactic-co-glycolic acid) (PLGA) nanoparticles, poly(lactic acid) (PLA) nanoparticles, poly(glutamic acid) (PGA) nanoparticles, polycaprolactone (PCL) nanoparticles, and polyester nanoparticles.
